(19) United States
(12) Patent Application Publication
(10) Pub. No.: US 2013/0030329 A1
(43) Pub. Date: Jan. 31, 2013

(54) APPARATUS AND METHODS FOR FRICTION REDUCTION

(71) Applicants: Jona Zumeris, Haifa (IL); Harold Jacob, Cedarhurst, NY (US)

(72) Inventors: Jona Zumeris, Haifa (IL); Harold Jacob, Cedarhurst, NY (US)

(21) Appl. No.: 13/521,060

(22) PCT Filed: Dec. 14, 2010
(86) PCT No.: PCT/US10/60159

§ 371 Date: Jul. 9, 2012

Related U.S. Application Data
(60) Provisional application No. 61/293,727, filed on Jan. 11, 2010.

(51) Int. Cl. A61H 23/02; A61N 7/00

(52) U.S. Cl. 601/2; 601/47

(57) ABSTRACT

An apparatus, system and methods thereof for generating an acoustic lubrication effect along a surface of an in-dwelling medical device that is in contact with a vital tissue in a subject are provided. The vibrations comprise: Hz range cylindrical surface vibrations; or kHz range surface acoustic wave (SAW) vibrations; or a combination thereof. The vibrations create an acoustic lubrication effect that may reduce pain or discomfort in a subject when the apparatus is used with an indwelling medical device. The vibrations may enhance tactile sensation when the medical device is a shoe pad.

### FIELD OF INVENTION

This invention relates to apparatus for generating vibrations along a surface of an indwelling medical device that is in contact with a vital tissue, wherein the vibrations create an acoustic lubrication effect along the surface of the indwelling medical device that is in contact with the vital tissue. More particularly, this invention relates to apparatus and methods thereof for reducing friction at an interface between an indwelling medical device and a vital tissue in a subject.

### BACKGROUND OF THE INVENTION

Minimally invasive medical procedures such as nasal gastric intubations and colonoscopies are widely used in medical practice. Despite the advantages of minimally invasive medical procedures, these therapies have various drawbacks including significant pain and discomfort.

Since its first description by Hunter in 1790, the NasoGastric tube (NG tube) has become one of the most frequently employed invasive devices used in hospitals. NG tube use is commonly associated with pain and discomfort at the nasal region (nose and face) and at the pharyngeal region. In addition, NG tube usage is associated with several complications: nasal ulcers, nasal septal injury, sinusitis, pharyngeal pain and tube clogging.

Currently available methods to alleviate pain and discomfort caused by minimally invasive medical procedures include use of lubricating jellies, with or without local anesthetics. These methods are only partially effective because the lubricating jelly is quickly absorbed into the surrounding tissues, e.g., nasopharyngeal tissue. A jelly coated invasive device therefore very quickly loses lubricity, the lubricated coating is quickly covered by a thin film of mucous and its lubricating ability is reduced. Thus lubricating jellies provide only a very short term solution.

Endoscopies, colonoscopies and other minimally invasive procedures involving the insertion of an endoscope into the colon of a patient are generally performed using intravenous (IV) sedation for alleviation of pain and discomfort. Undesirable effects of IV sedation drugs on a patient may include respiratory depression, anaphylaxis, other allergic reactions and/or missed work due to time off for recovery from drug effects.

The pain and discomfort of endoscopies are generally attributable to the stimulation of pain sensitive nerve endings found in the mucous membranes of the gastrointestinal tract. In attempts to alleviate the pain and discomfort in the absence of sedatives, oral and rectal local anesthetic sprays have been developed that deliver anesthetic agents to a particularly sensitive region. These agents may have only a nominal affect on the pain and discomfort experienced by the patient, as the endoscope is driven further into the gastrointestinal tract than the coverage of anesthetic sprays.

The manipulations used to move a colonoscope through the rectum and colon may be extremely painful and uncomfortable to a patient due to the nerve endings in the colon located in the mucosal and muscular walls. The principal forces to which these nerves are sensitive are stretching and tension, both of which occur when the relatively rigid colonoscope passes through the colon. In general, the only existing means of relieving the pain and discomfort that result from the stretching, torsion, and friction incurred during a colonoscopy is to provide sedation and analgesia to the patient, an often undesirable alternative since in addition to the above listed negative effects of sedation drugs, the drug levels required to relieve the discomfort of a colonoscopy often render the patient unable to cooperate during the procedure.

Analogous to the use of jelly lubricants for NG tube insertion, endoscopic devices have also been coated with a petrolatum or water-based lubricants prior to insertion, as a means of easing patient discomfort. However, in the case of colonoscopy, these lubricants are often removed from the colonoscope as it is inserted into the rectum and advanced through the anal sphincter. Very little lubricant remains afterwards to ease further manipulation of the colonoscope. Certain existing devices attempt to reduce the amount of lubricant and anesthetic lost during insertion of the colonoscope by employing a syringe or flexible plastic bottle equipped with a long applicator tip to coat the surface of the colonoscope while the colonoscope passes through the rectum. See, for example, U.S. Pat. No. 6,962,564. Though such devices may have some effect in reducing the friction coefficient of the colonoscope, much of the lubricant and/or anesthetic may still be lost as the colonoscope is pushed farther into the colon. Further, applying the lubricant and/or anesthetic to the colonoscope once inside the rectum, may fully coat the scope and may interfere with clear observations of tissue.

Past attempts have been made to make indwelling devices, e.g., NG tubes, colonoscopes and catheters, pass more easily through tubular body structures by coating them with polymers having low coefficients of friction. See for example, International PCT Application Publication No., WO2007089724 and U.S. Pat. No. 7,008,979. Such coatings must be bonded to the device, either covalently or by other means. These coatings are subject to wear and eventually can lose their effectiveness, particularly when applied to reusable devices such as colonoscopes and cystoscopes. Permanent coatings must also be able to withstand sterilization and/or disinfecting procedures without losing effectiveness, a difficult technical requirement. Further, such coatings may make devices quite slippery when wetted and therefore difficult for medical personnel to handle and properly manipulate.

Additional attempts to solve the problem of pain and discomfort during minimally invasive medical procedures and the indwelling of invasive devices have not overcome the ineffectiveness of lubrication jellies and coatings, or the negative effects of drug use.

For example, US Patent Application No. 20060282086 describes a device for introducing a nasogastric (NG) tube into the stomach of an anaesthetized or comatose patient, wherein relief from pain and discomfort is achieved only through lack of consciousness or use of drugs. U.S. Pat. No. 5,752,511 describes increasing patient comfort by nasal dilation technology. The mechanism described does not address pain associated with NG tube insertion and indwelling, and may in fact lead to increased irritation and pain of a patient's nose and surrounding facial tissue.

Innovations employing vibrations of various amplitudes and frequencies on an insertion device to disrupt bacterial growth (see US Patent Application No. 20070213645), to monitor placement of a device (see International PCT Application Publication No. WO 2005097246) and for disruption of blood vessel obstructions (see US Patent Application No. 20040138570) do not address or solve the problems of pain and discomfort experienced by the patient during minimally invasive medical techniques.

Endeavors to safely minimize the pain and discomfort associated with minimally invasive medical procedures have produced unsatisfactory results and are ineffective. Solutions are needed to meet the long-felt unmet medical need to safely alleviate the pain and discomfort experienced during minimally invasive medical procedures.

### SUMMARY OF THE INVENTION

In one embodiment, this invention provides an apparatus for generating vibrations along a surface of an indwelling medical device that is in contact with a vital tissue, the vibrations comprising: (a) Hz range cylindrical surface vibrations; or (b) kHz range surface acoustic wave (SAW) vibrations; or (c) a combination thereof, wherein the vibrations create an acoustic lubrication effect along the surface of the indwelling medical device that is in contact with the vital tissue.

In one embodiment, the acoustic lubrication effect reduces friction at an interface between the indwelling medical device and the vital tissue of a subject.

In one embodiment, the SAW vibrations create a rolling effect.

In one embodiment, the apparatus comprises an actuator electrically connected to an electronic driver, wherein the driver powers the actuator. In one embodiment, the driver comprises a power source. In one embodiment the power source is a battery, a microprocessor, or a firmware. In one embodiment, an electrical signal is generated by the driver, the signal controls the functionality of the actuator unit and the driver displays the functionality of the actuator. In one embodiment, the actuator receives electrical energy from the driver and the actuator responds by generating vibrations.

In one embodiment, the actuator comprises at least one vibration element. In one embodiment, the at least one vibration element comprise a piezo mechanical vibrator and/or an electro mechanical vibrator.

In one embodiment, the actuator is disposable. In one embodiment, the actuator is attached to the indwelling medical device. In one embodiment, the actuator is connected to a driver and attached to an indwelling medical device. In one embodiment, the actuator is attached to a non-invasive end of the indwelling medical device. In one embodiment, the attachment is via a clip-on stabilization mechanism. In one embodiment, the actuator may be repositioned while in use by sliding the actuator along the indwelling medical device either proximally or distally to an invasive end of the indwelling medical device.

In one embodiment, the piezo mechanical vibrator creates surface acoustic waves at about 90-120 kHz and cylindrical surface vibrations at about 30-70 Hz. In one embodiment, the electro mechanical vibrator creates cylindrical surface vibrations at about 70-200 Hz.

In one embodiment, the amplitude of the Hz range vibrations ranges between about 1 micron to 10 microns. In one embodiment, the amplitude of the kHz range surface acoustic wave vibrations ranges between about 0.1 micron and 1 micron. In one embodiment, the Hz range vibrations create acoustic lubrication with an amplitude between about 1 microns to 10 microns. In one embodiment that kHz vibrations create acoustic lubrication with an amplitude between about 0.1 micron and 1 micron.

In one embodiment, the Hz range vibrations, the kHz range vibrations or the combination of Hz range vibrations and kHz range vibrations generate pressure waves along the medical indwelling device.

In one embodiment, the indwelling medical device comprises: a nasal-gastric (NG) tube, a colonoscope, a gastroscope, a duodenscope, a bronchoscope, a cytoscope, a cystoscope, a urethroscope, a hemorrhoids treatment tube, a vaginal tube, an ultrasound scope, a catheter, a cauterizing tube, a cannula, a flexible endoscope or any other medical device utilized during minimally invasive procedures. In one embodiment, the indwelling device is a nasal-gastric tube (NG). In one embodiment, the indwelling medical device is a colonoscope. In one embodiment, the indwelling device is an ultrasound scope. In one embodiment, the indwelling medical device is disposable.

In one embodiment, the acoustic lubrication effect is a result of vibrations from a piezo mechanical vibrator or vibrations from an electro mechanical vibrator, or from combined vibrations from a piezo mechanical vibrator and an electro mechanical vibrator. In one embodiment, the acoustic lubrication effect reduces pain and/or discomfort in the subject. In one embodiment, the pain and/or discomfort is reduced during insertion, removal, indwelling or any combination thereof, of the indwelling medical device.

In one embodiment, the Hz range cylindrical surface vibrations or kHz range surface acoustic waves (SAW) or a combination thereof improve a quality of action of an implement that inserts into an inner channel of a medical indwelling device. In one embodiment, the implement comprises a polypectomy snare, a sphincterotome, a papillotome, a needle knife papillotome, or any other implement used for a trans-endoscopic electro surgery. In one embodiment the implement is disposable. In one embodiment, the quality of trans-endoscopic electro surgery is improved due to minimal injury of tissue.

In one embodiment, the Hz range cylindrical surface vibrations or kHz range surface acoustic waves (SAW) or a combination thereof improve definition of structures imaged using an indwelling device. In one embodiment, the indwelling device is an ultrasound scope.

In one embodiment, this invention provides an apparatus for generating micro-vibrations within a medical shoe pad, the vibrations comprising: (a) Hz range cylindrical surface vibrations; or (b) kHz range surface acoustic wave (SAW) vibrations; or (c) a combination thereof, wherein the vibrations enhance tactile sensation in a foot, dependent on changes in pressure created due to the vibrations. In one embodiment, the apparatus generating micro-vibrations is incorporated into a shoe pad or into medical shoe pad.

In one embodiment, the apparatus for generating micro-vibrations comprises an actuator and a driver. In one embodiment, the actuator comprises at least one vibration element comprising a piezo mechanical vibrator, an electro mechanical vibrator or a combination thereof. In one embodiment, the driver powers the apparatus by activating the actuator. In one embodiment, the driver powers a piezo mechanical vibrator and/or electro mechanical vibrator. In one embodiment, the apparatus is incorporated into a medical shoe pad. In one embodiment, the micro-vibrations are transferred from a piezo mechanical vibrator through a silicone layer of the shoe pad to the patient's foot.

In one embodiment, the apparatus for generating micro-vibrations within a medical shoe pad comprises an actuator and a driver, wherein the driver is electrically connected to the actuator and the driver powers the actuator, so that the actuator produces micro-vibrations, which comprise Hz range vibrations, kHz range surface acoustic wave (SAW) vibrations, or a combination thereof; and the micro-vibrations enhance tactile sensation in a foot.

In one embodiment, this invention provides a system comprising an apparatus for generating vibrations along a surface of an indwelling medical device that is in contact with a vital tissue, the vibrations comprising: (a) Hz range cylindrical surface vibrations; or (b) kHz range surface acoustic wave (SAW) vibrations; or (c) a combination thereof, wherein the vibrations create an acoustic lubrication effect along the surface of the indwelling medical device that is in contact with the vital tissue. In one embodiment, the system further comprises an indwelling tube, a display, a computer, a user board, an imaging system, handles, fixtures, stickers, adhesion pads, stands, timers, detectors, sensors, attachments or a combination thereof.

In one embodiment, this invention provides a method for generating an acoustic lubrication effect along a surface of an indwelling medical device that is in contact with a vital tissue, the method comprising use of an apparatus of this invention comprising an actuator electrically connected to an electronic driver to power the actuator, which comprises at least on vibration element for generating (a) Hz range cylindrical surface vibrations; or (b) kHz range surface acoustic wave (SAW) vibrations; or (c) a combination thereof, wherein the vibrations create an acoustic lubrication effect along the surface of the indwelling medical device that is in contact with the vital tissue.

In one embodiment of the method, the acoustic lubrication effect reduces friction at an interface between the indwelling medical device and the vital tissue of a subject.

In one embodiment of the method, the apparatus comprises a driver and an actuator. In one embodiment, the driver comprises a power source. In one embodiment of the method, the power source powers the actuator. In one embodiment of the method, the actuator comprises at least one vibration element. In one embodiment of the method, the at least one vibration element comprises a piezo mechanical vibrator or an electro mechanical vibrator. In one embodiment of the method, there are at least two vibration elements, the elements being a piezo mechanical vibrator and an electro mechanical vibrator. In one embodiment of the method, the actuator is connected to a driver and attached to an indwelling medical device. In one embodiment of the method, the attachment is via a clip-on stabilization mechanism. In one embodiment of the method, the actuator may be repositioned while in use by sliding the actuator along the indwelling medical device either proximally or distally to an invasive end of the indwelling medical device.

In one embodiment of the method, the piezo mechanical vibrator creates surface acoustic waves at about 90-120 kHz and cylindrical surface vibrations at about 30-70 Hz. In one embodiment of the method, the electro mechanical vibrator creates cylindrical surface vibrations at about 70-200 Hz. In one embodiment of the method, the amplitude of the Hz range vibrations ranges from about 1 micron to 10 microns. In one embodiment of the method, the amplitude of the kHz range surface acoustic waves' vibrations ranges between about 0.1 micron and 1 micron.

In one embodiment of the method, the indwelling medical device comprises: a nasal-gastric (NG) tube, a colonoscope, a gastroscope, a duodenscope, a bronchoscope, a cytoscope, a cystoscope, a urethroscope, a hemorrhoids treatment tube, a vaginal tube, an ultrasound scope, a catheter, a cauterizing tube, a cannula, a flexible endoscope or any other medical device utilized during minimally invasive procedures. In one embodiment of the method, the indwelling device is an ultrasound scope.

In one embodiment of the method, the acoustic lubrication effect is a result of vibrations from a piezo mechanical vibrator, vibrations from an electro mechanical vibrator, or from combined vibrations from a piezo mechanical vibrator and an electro mechanical vibrator. In one embodiment of the method, the acoustic lubrication effect reduces pain and/or discomfort in the subject. In one embodiment, the pain and/or discomfort is reduced during insertion, removal, indwelling or any combination thereof, of the indwelling medical device.

In one embodiment of the method, the Hz range cylindrical surface vibrations or kHz range surface acoustic waves (SAW) or a combination thereof improve a quality of action of an implement that inserts into an inner channel of the medical indwelling device In one embodiment of the method, the implement comprises: a polypectomy snare, a sphincterotome, a papillotome, a needle knife papillotome, or any other implement used for a trans-endoscopic electro surgery.

In one embodiment, methods for generating an acoustic lubrication effect along a surface of an indwelling medical device are used with a subject having a minimally invasive medical technique comprising insertion, removal, indwelling or any combination thereof of an to indwelling medical device.

In one embodiment, the minimally invasive medical technique in which the method for generating an acoustic lubrication effect along a surface of an indwelling medical device is used comprises: naso-gastric tube insertion; an endoscopic procedure comprising a colonoscopy, imaging technique, hemorrhoid treatment, trans-endoscopic electro surgery; insertion of a medical indwelling device; frigidity treatment; or any combination thereof.

In one embodiment of the method, Hz range vibrations or kHz range surface acoustic waves (SAW) or a combination thereof provide a means for reduced procedural time for minimally invasive medical procedures comprising insertion, removal, indwelling or any combination thereof of the indwelling medical device. In one embodiment of the method, the vibrations provide improved ergonomics of indwelling medical devices, thereby increasing efficiency and/or safety of use of the indwelling medical device. In one embodiment, the vibrations reduce bacterial adhesion on an inner channel of the indwelling medical device. In one embodiment of the method, the vibrations decrease impedance to electro cautery in the vital tissue. In one embodiment of the method, the vibrations improve definition of structures imaged using the indwelling device, wherein the indwelling device is an ultrasound scope.

### BRIEF DESCRIPTION OF THE DRAWINGS

The present invention will be understood and appreciated more fully from the following detailed description taken in conjunction with the appended drawings in which:

FIG. 1 is an illustration of a force diagram for a block on the ground;

FIGS. 2A and 2B are illustrations of contact area between an indwelling medical tube and a vital tissue in the absence (FIG. 2A) and presence (FIG. 2B.) of acoustic lubrication phenomenon;

FIG. 3 is a schematic illustration of cylindrical type acoustic wave propagation along a medical tube;

FIG. 4 is an illustration of a computer model view of acoustic lubrication along a medical tube. The vibration modes may consist of Hz vibrations (cylinder or hoop waves), or kHz surfaces acoustic wave vibrations or a combination thereof;

FIG. 5 is an illustration of surface acoustic waves' particle cylindrical motion;

FIG. 6 shows the generation of a compression wave in to a fluid (λ1) by a SAW (λ) at an angle (ξ) to the device surface;

FIGS. 7A, 7B, 7C and 7D illustrate mathematical simulations of acoustic wave vibrations traveling along a medical tube (7A-7C) and vibration mode dependent cross section views of a medical tube showing the changes in tube configuration depending on the vibration mode (7D);

FIG. 8 illustrates symmetrical and asymmetrical Lamb waves configurations;

FIG. 9 illustrates particle motion direction in Surface Acoustic Waves (SAW). SAW are the waves which travel between two surfaces. The particle moves in circular manner, therefore creates “rolling effect”;

FIG. 10 illustrates the acoustic lubrication effects of an actuator attached to a medical tube and the resultant acoustic wave propagation (10-40) along the surface of the medical device (10-10); and mathematical simulation of device diameter forms due to oscillations (10-50);

FIG. 11 is a schematic diagram of an apparatus for generating vibrations along a surface of an indwelling medical device in use, consisting of an actuator (11-1) connected via a cable (11-2) with a driver (11-3) applied to a medical tube (11-4) which is inserted into the patient's nasal-gastric tract (11-5);

FIG. 12 illustrates acoustic lubrication actuator incorporated into NG tube holder and attached to patient's nose;

FIG. 13 shows an actuator clipped to standard nasal-gastric tube and connected with a cable to the driver;

FIG. 14. shows an actuator for generating acoustic lubrication attached to NG tube;

FIG. 15 shows an electronic driver unit that may power the actuator for generating acoustic lubrication;

FIGS. 16A, 16B, 16C and 16D show acoustic lubrication actuator placement procedure;

FIG. 17 is a schematic illustration of the driver system element functions;

FIG. 18 shows a detailed actuator design, wherein vibration element 18-10 and vibration element 18-20 are incorporated in the actuator case 18-40 and connected via cable 18-90 to a driver (not shown), which activates the actuator and results in acoustic lubrication along a tube surface;

FIG. 19 shows a schematic representation of the actuator's vibration mechanism;

FIG. 20 schematically illustrates the actuator attached to a tube and acoustic wave propagation on the tube surfaces, resulting in the generation of the acoustic lubrication effects;

FIG. 21 shows a measurement set up for acoustic lubrication system attached to colonoscope;

FIG. 22 shows an electro surgery accessory (an implement) inserted into a colonoscope inner channel; Vibrations may have an impact on polyps surgery procedure ease;

FIG. 23 schematically illustrates an acoustic lubrication system for colonoscope;

FIG. 24 shows the principle schema of disposable, clipped-on actuator developing acoustic lubrication on colonoscope surfaces;

FIG. 25 shows amplitude measurements and pressure calculation results on an NG tube with amplitude measurement set up, as shown in FIG. 26 and pressure calculations provided in the examples;

FIG. 26 illustrates the set up for measuring vibration amplitudes and frequency measurements on an NG tube; The study was conducted with MTI-2000 Fotonic Sensor, when digital display operating in the volts mode was calibrated for the probe gap vs. output signal (analog displacement signal-voltage signal was converted to amplitudes mm);

FIG. 27 illustrates measurement results of an impact of acoustic lubrication applied to NG tube;

FIG. 28 graphically illustrates clinical results showing reduction of discomfort level during indwelling phase of a nasal-gastric tube;

FIG. 29 graphically illustrates clinical results showing reduction of pain level during indwelling phase of nasal-gastric tube;

FIGS. 30A, 30B and 30C show a device for frigidity treatment in women and for enhancing sexual intercourse;

FIGS. 31A and 31B show a device for decreasing hemorrhoid pain due to micro-massage and acoustic lubrication;

FIGS. 32A, 32B and 32C show acoustic micro-massaging shoe inserts creating micro-massage effects on the feet, thereby relieving balance problems common in an elderly population;

FIGS. 33A, 33B, 33C, 33D, 33E, 33K, 33L, 33M and 33N, illustrate different actuator attachment methods for creating desired vibration modes on medical tubes.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention is directed to an apparatus for reducing friction between an indwelling medical device and a vital tissue that the indwelling medical device contacts and to methods of use thereof.

Specifically, the present invention may be used to reduce pain and/or discomfort of minimally invasive medical procedures that utilize indwelling medical devices. The present invention may also be used to improve functionality of indwelling medical devices. These uses are not mutually exclusive. The apparatus, principles, system and methods of use according to the present invention may be better understood with reference to the drawings and accompanying descriptions.

Before explaining at least one embodiment of the present invention in detail, it is to be understood that the invention is not limited in its application to the details of construction and the arrangement of the components as set forth in the following description or illustrated in the drawings. The invention is capable of other embodiments or of being practiced or carried out in various ways. Also, it is to be understood that the phraseology and terminology employed herein are for the purpose of description and should not be regarded as limiting.

### I. BASIC PRINCIPALS

### Theoretical Basis of Friction

Friction is not a fundamental force, as it is derived from electromagnetic forces between charged particles, including electrons, protons, atoms, and molecules, and so cannot be calculated from first principles, but instead must be found empirically.

When contacting surfaces move relative to one another, the friction between the two surfaces converts kinetic energy into thermal energy, or heat. Contrary to earlier explanations, kinetic friction is now understood not to be caused by surface roughness but by chemical bonding between the surfaces. Surface roughness and contact area, however, do affect kinetic friction for micro- and nano-scale objects where surface area forces dominate inertial forces.

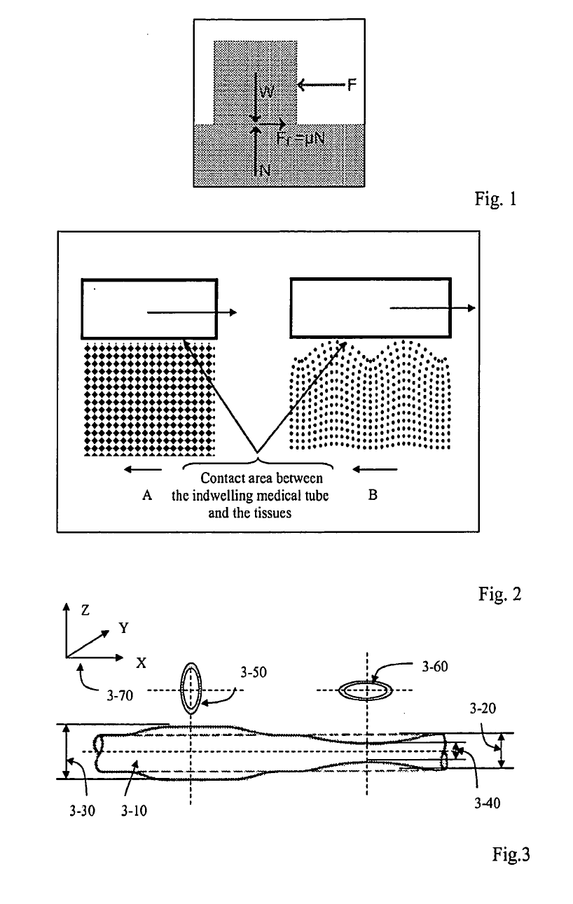

FIG. 1 uses a Force Diagram for a block on the ground to illustrate the basic forces involved in friction. Vectors indicate directions and magnitudes of forces. W is the force of weight, N is the normal force, F is an applied force of unidentified type, and Ff is the force of kinetic friction, which is equal to the coefficient of kinetic friction μ times the normal force, μN. Since the magnitude of the applied force is greater than the magnitude of the force of kinetic friction opposing it, the block is moving to the left.

Dry friction resists relative lateral motion of two solid surfaces in contact. Dry friction is also subdivided into static friction between non-moving surfaces, and kinetic friction (sometimes called sliding friction or dynamic friction) between moving surfaces.

Rolling friction (sometimes called rolling resistance or rolling drag) is the resistance that occurs when a round object such as a ball or tire rolls on a flat surface. It is caused mainly by the deformation of the object, the deformation of the surface, or both. Additional contributing factors include wheel radius, forward speed, surface adhesion, and relative micro-sliding between the surfaces of contact. It depends very much on the material of the wheel or tire and the sort of ground.

Rolling friction is also described by the resistance which one body offers to another when rolling along its surface, the two being pressed together by some force. This resistance, like that in sliding friction, arises from the inequalities of two surfaces. The coefficient of rolling friction is equal to the quotient obtained by dividing the entire force of friction by normal pressure.

The coefficient of rolling friction is generally much less for tires or balls than the coefficient of sliding friction.

The following laws of friction have been established when a cylindrical body or wheel rolls upon a plane:

1. The coefficient of rolling friction is proportional to the normal pressure;

2. The coefficient of rolling friction is inversely proportional to the diameter of cylinder or wheel;

3. The coefficient of rolling friction increases as the surface of contact and velocity increase.

In many cases there is a combination of both sliding and rolling friction in the same machine. For example, rubber will give a bigger rolling resistance than steel. Also, sand on the ground will give more rolling resistance than concrete. A moving wheeled vehicle will gradually slow down due to rolling resistance including that of the bearings, but a train car with steel wheels running on steel rails will roll farther than a bus of the same mass with rubber tires running on pavement. Hard wheels rolling on and deforming a soft surface, results in the force from the surface having a component that opposes the motion of the wheels.

### II. DEFINITIONS

As used herein, the term “medical indwelling device” refers to any medical device implanted or inserted in the human body. Such devices may be temporarily implanted or inserted. In one embodiment, implantation or insertion time is less than a minute, for example insertion of a needle. In one embodiment, a medical device may be temporarily implanted or inserted for a time period of months, as may be for implantation or insertion of urinary catheters. In other embodiments, implantation or insertion is for a time period between a minute and months. In one embodiment, the time period of implantation or insertion is dependent upon the indwelling device being implanted or inserted and the needs of the subject.

The term “medical indwelling device” may also be referred to herein as a “tube”, a “medical tube” or an “indwelling device”. In this, context a “tube”, a “medical tube” or an “indwelling device” have all the qualities and properties of a medical indwelling device.

As used herein, the term “interface” refers to the surface forming a common boundary of two bodies. This boundary may be the place at which independent and often unrelated systems meet and act on each other, e.g., an indwelling medical device and a vital tissue.

As used herein, the term “acoustic lubrication” refers to elastic wave propagation on a tube surface, which compresses and expands the cross section of the tube, therefore reducing the contact area and time between the two surfaces, i.e., between an indwelling medical device and a vital tissue. Thus, the elastic wave propagation acts as an “acoustic lubricant” that lessens or prevents friction or difficulty in moving a device through a cavity of the body (for example colonoscope to move through the colon)

As used herein, the term “acoustic lubrication effect” refers to the resultant acoustic lubrication along the surface of a medical indwelling device. In one embodiment, the acoustic lubrication effect reduces friction between an indwelling device and a vital tissue of a subject, as it relates to the use of an indwelling medical device. In one embodiment, the acoustic lubrication effect reduces pain experienced by a subject, reduces discomfort experience by a subject, or any combination therein, as they relate to the use of an indwelling medical device. Further, the term “acoustic lubrication effect” refers to an acoustic lubrication effect resultant from Hz range vibrations (cylindrical surface vibrations) or kHz range SAW vibrations (a rolling effect) or a combination thereof. As used herein, the term “acoustic lubrication effect” may also be referred to herein as “acoustic lubrication effect and a rolling effect”. An “acoustic lubrication effect” may be generated by means of an electromechanical vibrator or by means of a piezoelectric vibrator or by a combination thereof.

As used herein, the term “combination” may also be referred to herein as “complex”

As used herein, the term “surface” refers to an organic surface or an inorganic surface. An organic surface may for example be skin, surgical sutures, a mucosal membrane surface, an epithelial surface or a surface of a vital tissue. An inorganic surface may for example be an exterior or upper boundary of an object or body, for example the outer surface of a tube. An inorganic surface may be a plane or curved two-dimensional locus of points, as the boundary of a three-dimensional region.

As used herein, the term “contact” refers to the spatial relationship between a surface of a medical indwelling device and a vital tissue. Contact may be constant, uniform, persistent, continuous or momentary, irregular, non-uniform, discontinuous. Contact may be of equal or unequal values and extent. Contact between an indwelling medical device and a vital tissue may have any combination of characteristics as described herein.

As used herein, the term “contact time” refers to the period of time the tube is in contact with a vital tissue. Contact time may be measured in milliseconds, seconds, minutes, hours, days or any combination of length of time therein. Contact time may be reduced by about 50%. Contact time may refer to any given point along a tube at any given time.

As used herein, a “tissue” refers to an aggregation of cells of one or more cell types which together perform one or more specific functions in an organism.

As used herein, a “tissue surface” refers to that portion of a tissue that forms a boundary between a given tissue and other tissues or the surroundings of the tissue. A tissue surface may refer to an external surface of an animal, for example the skin or cornea, or, alternatively, the term may refer to a surface that is either internal, for example, the lining of the gut, or to a surface that is exposed to the outside surroundings of the animal only as the result of an injury or a surgical procedure.

As used herein, a “vital tissue” refers to living tissue.

As used herein, “firmware” refers to the fixed, usually small programs and data structures that internally control various electronic devices.

As used herein, the terms “minimally invasive medical procedure” or “minimally invasive procedure” refers to any procedure (surgical or otherwise) that is less invasive than open surgery used for the same purpose. A minimally invasive procedure typically involves use of laparoscopic devices and remote-control manipulation of instruments with indirect observation of the surgical field through an endoscope or similar device. Minimally invasive procedures may be carried out through the skin or through a body cavity or anatomical opening. This may result in shorter hospital stays, or allow outpatient treatment. (Wickham JEA. The new surgery. Br Med J 1987; 29:1581-1582) When there is minimal damage of biological tissues at the point of entrance of instrument(s), the procedure is called minimally invasive. Minimally invasive procedures of the proposed invention may include: indwelling catheterization (urinary, intravascular, naso gastric, etc.), endoscopy, percutaneous surgery, laparoscopic surgery, arthroscopic surgery, cryosurgery, microsurgery, endovascular surgery (such as angioplasty), and coronary catheterization.

In one embodiment, minimally invasive techniques are used for insertion of a medical catheter (such as urinary or intravascular, or any other indwelling medical catheter) or nasal gastric intubation, diagnostics, treatment and/or drug-delivery. In one embodiment, treatment comprises an endoscopic medical procedure. In one embodiment, treatment comprises a medical operation or medical procedure involving electrocautery. In one embodiment, the operation or medical procedure involve frigidity treatment in women. In one embodiment, the medical procedure involves decreasing hemorrhoid pain. In one embodiment, the medical procedure involves imaging.

As used herein, the term “actuator” may also be referred to herein as a “vibrato” or “vibrator element”. In this context a “vibrato” or a “vibrator element” has all the qualities and properties of an actuator. In one embodiment, an actuator comprises at least one vibrator or at least one vibrating elements.

As used herein, the term “attached” refers to the joining, adhering, connecting or the like of at least two elements. Two elements will be considered attached together when they are attached directly or indirectly to one another. By indirect attachment is meant, when each element is directly attached to intermediate elements. In one embodiment, the attachment between elements is stable, wherein the attachment is such as to resist forces tending to cause motion or change of motion. In one embodiment, the attachment between elements is temporary. In one embodiment, the attachment between elements is permanent.

As used herein, the term “vibration(s)” may also be referred to herein as “micro-vibration(s)” or “wave(s)” having all the same qualities and properties. Vibrations are defined by their frequency and by their amplitude.

Hz is a unit of frequency and is defined as the number of complete cycles per second.

In one embodiment, vibration amplitude of Hz vibrations ranges between 1 to 10 microns. In one embodiment, the vibration amplitude of kHz vibrations ranges between 0.1 and 1 micron. In one embodiment, the amplitude unit used is micrometers, wherein as used throughout, the meaning of micrometer is identical to the meaning of a micron.

As used herein, the term “micro massage effects” refers to mechanical stimulation of cells due to employing pressure exchanges which occur from vibrations. The amplitudes of such vibrations are equal to or smaller than micron range amplitude measurements.

As used herein, the term “surface acoustic waves” or “SAW” includes several types of waves or combinations thereof, as follows:

1. Surface-Rayleigh (elliptical orbit-symmetrical mode);

2. Plate Wave-Lamb-component perpendicular to surface (extensional wave);

3. Plate Wave-Love-parallel to plane layer, perpendicular to wave direction;

4. Stoneley (Leaky Rayleigh Waves)-wave guided along interface; and

5. Sezawa-antisymmetric mode.

As used herein, the term “pressure” refers to a force per unit area.

Periodic motion causes pressure waves in surrounding physical media. Sound waves are made of high pressure and low pressure pulses traveling through a medium. The high pressure areas (compression) are where the particles have been squeezed together; the low pressure areas (rarefaction) are where the particles have been spread apart. The wavelength of sound is the distance between two successive high pressure pulses or two successive low pressure pulses.

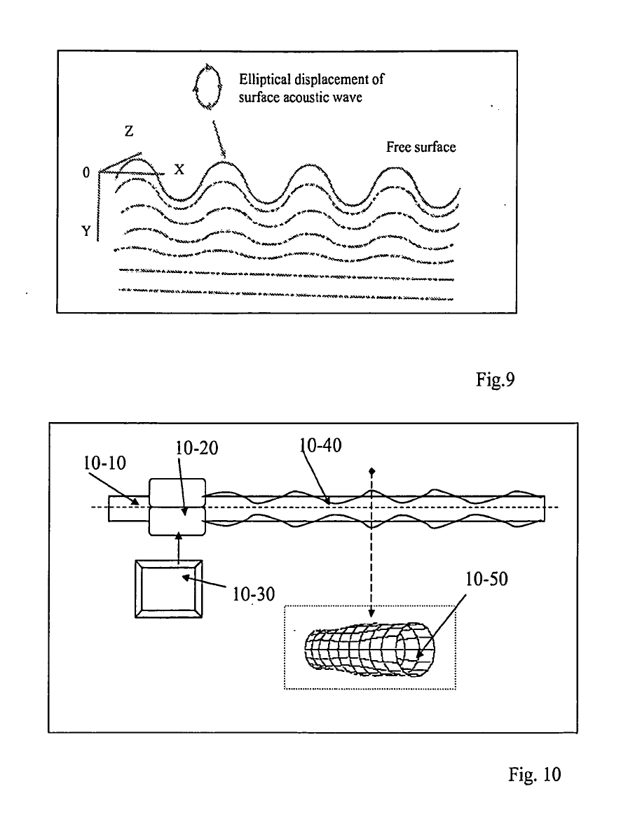

FIG. 9 illustrates particle motion of surface acoustic waves.

As used herein when describing the connection between an actuator and a driver, the term “connect” refers to an electrical connection between an actuator and a driver.

As used herein, the term “temporary” or “temporarily” refers to a limited time period. In one embodiment, the time period is the duration of a surgical and/or implantation procedure. In one embodiment, the time period extends longer than the duration of the surgical and/or implantation procedure. In one embodiment, the time period is the duration of need of a subject.

In one embodiment, rolling effect occurs when waves travel between two surfaces and the wave particles move in circular manner.

In one embodiment, a medical indwelling device is a tube. In one embodiment, the medical indwelling device comprises a tube. In one embodiment, the tube has at least two ends. In one embodiment, the tube comprises an invasive end and a non-invasive end. In one embodiment, the tube is hollow. In one embodiment, the tube has an inner channel. In one embodiment, an implement may be inserted into or through the inner channel. In one embodiment, examples for implements may be a polypectomy snare, a sphincterotome, a papillotome, a needle knife papillotome, or any other implement used for a trans-endoscopic electro surgery. In one embodiment, implements or portions thereof may be positioned temporarily or permanently outside the invasive end of the tube.

In one embodiment, methods and apparatus of this invention decrease impedance to electro cautery. In one embodiment, reducing impedance to electro-cautery means easier and safer manipulations by the medical practitioner. In one embodiment, reducing impedance for electro-cautery is important in situations because less or no blooding appears as a result of the surgery with addition of micro vibrations on the surface of surgery tool.

In one embodiment, a medical shoe pad comprises an apparatus for generating micro-vibrations. In one embodiment, a medical shoe pad is used to enhance tactile sensation in a foot.

In one embodiment, a medical shoe pad comprises an apparatus for generating micro-vibrations wherein the apparatus comprising an actuator and a driver electrically connected so that the driver may power the actuator; and wherein the actuator produces micro-vibrations comprising Hz range vibrations, kHz range surface acoustic wave (SAW) vibrations, or a combination thereof; and the micro-vibrations enhance tactile sensation in a foot in contact with the medical shoe pad.

### III. APPARATUS AND SYSTEMS FOR REDUCTION OF FRICTION

If the friction between the surfaces of a vital tissue and an indwelling medical device could be reduced, the indwelling device could be inserted more smoothly, safely and with reduced pain and/or discomfort during indwelling.

In one embodiment, this invention provides an apparatus for generating vibrations along a surface of an indwelling medical device that is in contact with a vital tissue, the vibrations comprising: (a) Hz range cylindrical surface vibrations; or (b) kHz range surface acoustic wave (SAW) vibrations; or (c) a combination thereof, wherein the vibrations create an acoustic lubrication effect along the surface of the indwelling medical device that is in contact with the vital tissue. The acoustic lubrication effect may be the result of generation of cylindrical shape acoustic waves of different wave lengths being propagated along the tube surface.

In one embodiment, the apparatus comprises an actuator electrically connected to an electronic driver, wherein the driver powers the actuator; and wherein the actuator comprises at least one vibration element that produces vibrations comprising Hz range cylindrical surface vibrations, kHz range surface acoustic wave (SAW) vibrations, or a combination thereof; the vibrations create an acoustic lubrication effect along the surface of the indwelling medical device that is in contact with the vital tissue; and the acoustic lubrication effect reduces friction at an interface between the indwelling medical device and the vital tissue of a subject.

In one embodiment, vibrations are propagated along the entire length of a tube. In one embodiment, vibrations are propagated along a portion of the tube length less than the full length of the tube. In one embodiment, the tube acts as wave guide.

In one embodiment, the acoustic lubrication effect reduces friction at an interface between the indwelling medical device and the vital tissue of a subject.

Friction is the force resisting the relative motion of two surfaces in contact. In some embodiments of the invention the two surfaces in contact are (1) a surface of an indwelling medical device, for example a NasoGastric (NG) tube and (2) a surface of a vital tissue of a subject that the indwelling device contacts, such as nasal and/or pharyngeal tissues in contact with the (NG) tube.

In one embodiment, an indwelling medical device is a nasal-gastric tube (NG). A NG tube is an example of an indwelling medical device inserted nasally into the upper gastrointestinal tract.

In one embodiment, a subject is a human. In one embodiment a subject is a patient.

NG tube insertion and removal when the NG tube is in relative motion to nasal and pharyngeal tissues, causes friction but only for a brief period of time. A great deal of NG tube related pain and discomfort may occur during the indwelling phase, when the NG tube does not move. In this case the friction process is generated due to movements of nasal and pharyngeal tissues. These movements are created due to natural body movements such as swallowing, speaking, neck movements and breathing. It is known from the literature that these movements have a frequency range of 1-500 Hz.

Friction between two surfaces may be reduced with acoustic lubrication effects, with rolling effects or a combination thereof. In one embodiment, SAW vibrations create a rolling effect.

The inventive apparatus for reducing friction generates vibrations consisting of Hz vibrations or kHz vibrations or a combination thereof. Alone or in combination, these vibrations result in an acoustic lubrication effect that reduces the coefficient of friction between vital tissues in contact with an indwelling medical device.

Though acoustic lubrication is a well known concept and is widely used in various technical processes wherein one of the two interfaces is vibrated with frequencies in the 20 Hz-50 kHz range (K. Platenberg (1998) PhD dissertation, Wayne State University, Detroit, Mich.; Yoshinaka, K. et al., (2007) Tribology International 40:339-344), this effect has not been applied to decrease friction of medical indwelling devices.

Vibrational energy may be transmitted along an inorganic surface of the invention. Additionally, vibrational energy may be transmitted along an inorganic surface of the medical tube while the apparatus is attached to the medical tube, which is in contact with a vital tissue of a subject.

In one embodiment of the invention, contact between an indwelling medical device and vital tissue is constant. In one embodiment of the invention contact between an indwelling medical device and vital tissue is uniform. In one embodiment of the invention contact between an indwelling medical device and vital tissue is persistent. In one embodiment of the invention contact between an indwelling medical device and vital tissue is momentary. In one embodiment of the invention contact between an indwelling medical device and vital tissue is irregular. In one embodiment of the invention contact between an indwelling medical device and vital tissue to is non-uniform. In one embodiment of the invention contact between an indwelling medical device and vital tissue is discontinuous.

In one embodiment of the invention contact between an indwelling medical device and vital tissue is of equal value and extent. In one embodiment of the invention contact between an indwelling medical device and vital tissue is of unequal value and extent.

The present invention reduces contact time between an indwelling medical device and a vital tissue.

In some embodiments of the invention, wherein only low frequency Hz range cylindrical surface acoustic vibrations are used, tube-tissue contact time is reduced by 30-50%. In some embodiments of the invention, wherein a combination of low frequency Hz range cylindrical surface vibrations and kHz frequency SAW vibrations are used, tube-tissue contact time is reduced by 35-75%.

In one embodiment, tube-tissue contact time is reduced by more than 10%. In one embodiment, tube-tissue contact time is reduced by more than 20%. In one embodiment, tube-tissue contact time is reduced by more than 30%. In one embodiment, tube-tissue contact time is reduced by more than 40%. In some embodiments of the invention, wherein a complex of low frequency Hz range vibrations and kHz frequency SAW vibrations is used, tube-tissue contact time is reduced by 30-80%. In some embodiments of the invention, wherein a complex of low frequency Hz range vibrations and kHz frequency SAW vibrations is used, tube-tissue contact time is reduced by 20-90%. In some embodiments of the invention, wherein a complex of low frequency Hz range vibrations and kHz frequency SAW vibrations is used, tube-tissue contact time is reduced by 40-70%.

Acoustic lubrication occurs when acoustic waves are introduced between sliding surfaces, theoretically, causing them to be in contact for half of the time, hence substantially reducing friction. FIG. 2 illustrates an acoustic lubrication effect, wherein 2A represents the case when two surfaces are moving respectively one to another resulting in a force of friction between the surfaces; 2B illustrates a similar phenomenon of surfaces moving respectively in opposite directions from one another but in this instance one of the surfaces is vibrated. One can observe that the contact time and contact area between two moving surfaces is reduced, which results in a reduction of friction.

The present invention provides an apparatus for reducing friction at an interface between an indwelling medical device and a vital tissue, comprising an apparatus comprising. a (1) driver comprising a programmable electronic unit, and (2) an actuator comprising vibrator elements, wherein the actuator is electrically connected to the driver. Further, the actuator may be attached, for instance clipped onto, a medical indwelling device (for example, a nasal gastric tube, a colonoscope, or a cystoscope) in order to create the desired acoustic lubrication effect and rolling effect along the indwelling device and thereby reduce friction at the interface between the indwelling device and vital tissue.

In exemplary embodiments of the invention, the apparatus comprises a driver and an actuator, wherein the driver is connected to the actuator. In one embodiment, the connection between a driver and an actuator is electrical. The driver comprises an electronic unit for providing electrical signals to the actuator, and a power source. In one embodiment, the driver comprises an electronic unit, a power source and a microprocessor creating firmware. In one embodiment, the power source is a battery.

In one embodiment, the actuator comprises at least one vibration element. The at least one vibration element may be an electro-mechanical vibration element and/or piezo mechanical vibrator, that converts the electrical signal from the driver into mechanical vibrations. When an actuator is attached to a medical indwelling device, the actuator creates an acoustic lubrication effect along the tube.

The actuator receives electrical signals from the driver. When the driver is turned on, the actuator mechanically vibrates and generates acoustic waves that propagate along the tube surface. These acoustic waves produce the vibrations on the tube surface and create acoustic lubrication between the tube and tissues; therefore the contact time between the tube and the tissue is reduced, as illustrated in FIG. 2.

In exemplary embodiments of the invention, the actuator comprises at least one vibration element, which generates electro mechanical vibrations and/or piezo mechanical vibrations. In other exemplary embodiments, the actuator comprises at least two vibration elements, which generate electro mechanical vibrations and/or piezo mechanical vibrations.

In one embodiment, the electro-mechanical vibration element can generate low frequency cylindrical surface vibrations in 70-200 Hz range, which result in acoustic lubrication effect.

In one embodiment, the piezo mechanical vibration element generates kHz frequency surface acoustic waves (SAW), which create an acoustic lubrication rolling effect due to elliptical motion of surface particles—specific feature of surface acoustic waves. In one embodiment, a piezo mechanical vibration element is capable of creating 30-70 Hz range vibrations, which could not be achieved with electro mechanical vibrator. These waves are sufficient due to possibility to create longer wave length, when it is needed to reach the long tube end. In one embodiment, the piezo mechanical vibration element generates vibrations in a range from 90 kHz to 120 kHz. In another embodiment, the piezo mechanical vibration element generates a combination of vibrations at about 30-70 Hz and at about 90 kHz to 120 kHz. In one embodiment, the piezo mechanical vibrator creates vibrations at about 90-120 kHz and/or 30-70 Hz.

As used herein, “Hz range cylindrical surface vibrations” may also be referred to herein as “low frequency vibrations”, “Hz range vibrations”, “Hz frequency vibrations” or “cylindrical surface vibrations” having all the qualities and properties of Hz range cylindrical surface vibrations.

As used herein, “kHz surface acoustic wave vibrations” may also be referred to herein as “SAW vibrations”, “SAW”, “kHz frequency surface acoustic waves”, “kHz frequency surface acoustic waves vibrations”, “kHz frequency vibrations” or “kHz range vibrations” having all the qualities and properties of kHz surface acoustic wave vibrations.

When the actuator element vibrates, mechanical vibration energy is transmitted along the tube surface through the contact points between the actuator and a medical indwelling tube, as shown in FIG. 3. The actuator's vibrations are such that they generate cylindrical shape acoustic waves with different wave length propagation on the tube surface through its length. In this case, the tube acts as wave guide.

The shape of the cylindrical type acoustic wave is illustrated in FIG. 3. It should be understood, that the NG tube does not move while the tissue that it is in contact with continues to move during respiration, speech and subject movement. The propagation of cylindrical acoustic waves result in compression and extension of the tube, therefore the tube's cross section diameter changes in shape. FIG. 3 is a schematic illustration of cylindrical type acoustic wave propagation on the tube: 3-10—NG tube; 3-20—the nominal diameter of NG tube; 3-30 and 3-40—maximal and minimal tube cross section; 3-50 and 3-60 cross-sections of the NG tube alternately compressed and expanded in perpendicular directions; 3-70—coordinate system.

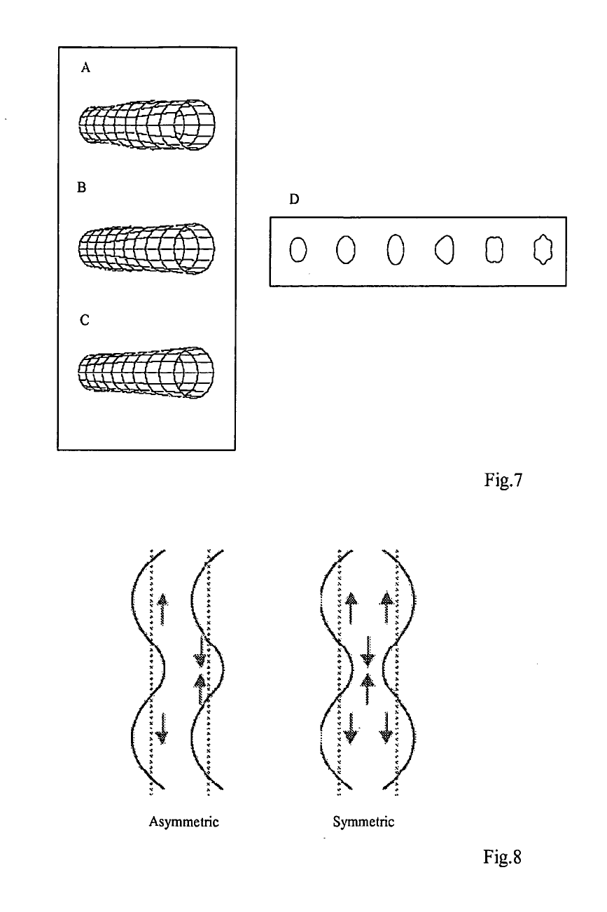

Reference is now made to FIG. 7A-7D, illustrating a mathematical simulation of acoustic waves traveling along the tube, (FIG. 7A-7C) and vibration mode dependent cross section views (FIG. 7D) of the tube illustrating the changes in tube configuration depending on vibration mode.

In order to strengthen the effect of reduced friction, a rolling effect is added to the system as illustrated in FIG. 5. The rolling effect is created with surface acoustic waves (SAW) generated on the surface of the indwelling medical device. Surface acoustic waves may be created by one of the methods disclosed in the United States Patent Application Publication No. 20050268921, (Zumeris et al “Nanovibration coating process for medical devices using multi vibration modes of a thin piezo element”), which is herein incorporated by reference.

The main quality of SAW is that surface material particles are caused to vibrate in cycles. Therefore a rolling effect is created. Working together the Hz vibrations' acoustic lubrications and rolling effect of surface acoustic waves create acoustic lubrication, thereby effectively reducing friction between an indwelling medical device and a vital tissue in contact with this device.

Surface or Rayleigh waves travel along the boundary between two different media and penetrate to a depth of about one wavelength. The particle movement has an elliptical orbit. Lamb wave is a special case of Rayleigh waves, which occurs when the material is relatively thin.

Reference is now made to FIG. 8 illustrating Rayleigh-Lamb waves. Rayleigh-Lamb waves are complex vibrational waves that travel through the entire thickness of a material. Propagation of Lamb waves depends on medical indwelling device material density, elasticity, and components. Lamb waves are influenced greatly by frequency selected and material thickness. With Lamb waves, a number of modes of particle vibration are possible, but the two most common are symmetrical and asymmetrical. The motion of the particles is similar to the elliptical orbits for all kinds of surface waves.

In some embodiments of the invention, there is provided an apparatus, comprising an actuator which may propagate acoustic waves of the “cylindrical” type along the tube surface. Cylindrical type is related to low frequency Hz range acoustic waves created with electromechanical vibrator.

Due to acoustic lubrication vibrations that are propagated on the tube surface, the surface points are oscillating with predetermined frequency and displacement amplitudes. The oscillating points create pressure at the contact points with tissue. Therefore, safety considerations are related to the vibration amplitudes, frequency or pressure. In one embodiment, the vibration amplitudes, frequency and/or pressure are all considered safe. See, for example, FIG. 25 which shows pressure calculation results along a tube. In one embodiment of this invention, the Hz range vibrations, the kHz range vibrations or the combination of Hz range vibrations and kHz range vibrations generate pressure along the medical indwelling device.

Methods described herein are based on exemplary embodiments of the invention in which a system is designed for an indwelling medical device being a NG tube or a colonoscope. In an exemplary embodiment, the indwelling medical device is an NG tube.

In addition in some embodiments of the invention, an apparatus is provided for hemorrhoid treatment and women's sexual treatment devices/toys, when their design principles include reduction of friction between vital tissues and a surface of the device, wherein the propagation of vibrations along such a device may decrease pain (in the case of hemorrhoids) or cause enhanced sexual feelings (in the case of sexual treatment).

The acoustic lubrication vibration waves are also intended to reduce the adhesion effect of the tube on the nasopharyngeal region. This adhesion effect contributes to the negative symptoms during NG tube usage.

In some embodiments of this invention, an indwelling medical devices comprises a NG tube, colonoscope, a flexible endoscopes, a gastroscope, a duodenscope, a bronchoscope, a cytoscope, a cystoscope, a urethroscope, a vaginal tube, a hemorrhoids treatment tube, an ultrasound scope, a catheter, a cauterizing tube, a cannula, or any other medical device utilized during minimally invasive procedures, wherein acoustic lubrications along their surfaces may be created by applying the principles described herein.

In some embodiments of the invention, the indwelling medical device is disposable.

Due to vibrations created with electro mechanical and/or piezo mechanical vibrator elements, comprised in an actuator, acoustic waves propagate on the indwelling medical device and reduce the friction coefficient at points of contact between the indwelling medical tube and the vital tissues during the period of tube usage.

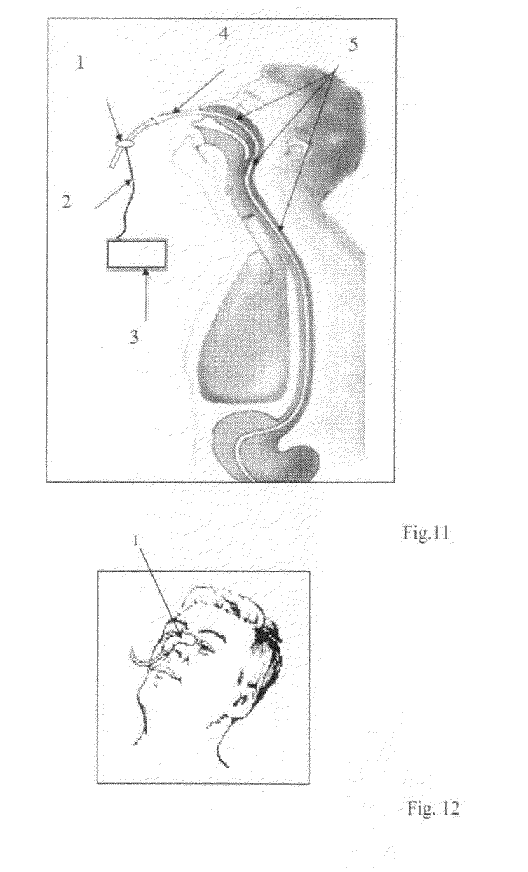

FIGS. 11 and 12 illustrate an exemplary apparatus of the invention, wherein an actuator is attached to an NG tube, which is further attached and inserted into a patient's nose. In these illustrations, the actuator may produce kHz range surface acoustic wave vibrations and create a “rolling effect”, because the actuator is placed nearby to the tube entrance into the body. Therefore, in one embodiment of the invention, it may be enough to use one component only, i.e. kHz range SAW vibrations, out of the possible combinations of vibrations.

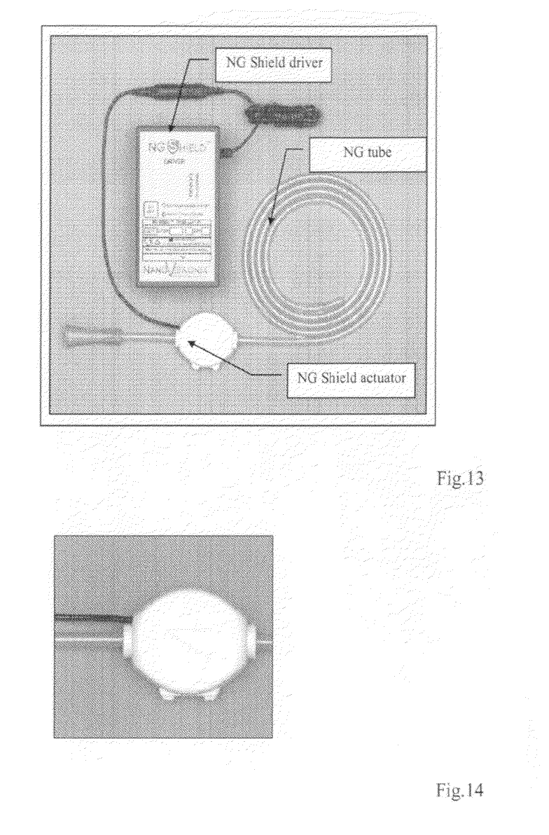

The photograph in FIG. 13 shows an actuator clipped to a standard nasogastric tube connected with a cable to the driver.

In one embodiment of the invention, the driver comprises a power source, a microprocessor and firmware. In another embodiment of the invention, the power source may be a battery or multiple batteries. In one embodiment, the driver unit contains batteries to power the apparatus, the microprocessor (CPU) and the firmware to generate the electrical signal, which controls and displays the functionality of the actuator unit. The driver is small and lightweight. The cable provides an electrical connection between the driver and the actuator. In one embodiment, the driver powers an actuator using at least one battery contained within the driver.

In one embodiment, the attachment of an actuator to a medical indwelling device is via a clip-on actuator. A clip-on actuator of the invention, which may be disposable, is a small and lightweight device that is attached to the medical tube via a clip-on stabilization mechanism. In one embodiment, the actuator is disposable.

The actuator receives electrical energy from the driver and responds by creating mechanical displacements, thus generating low frequency cylindrical type acoustic wave propagation on the tube surface and/or simultaneous SAW waves. These vibration waves, alone or in combination, generate the effective friction reduction between the NG tube surface and the surrounding nasopharyngeal tissues.

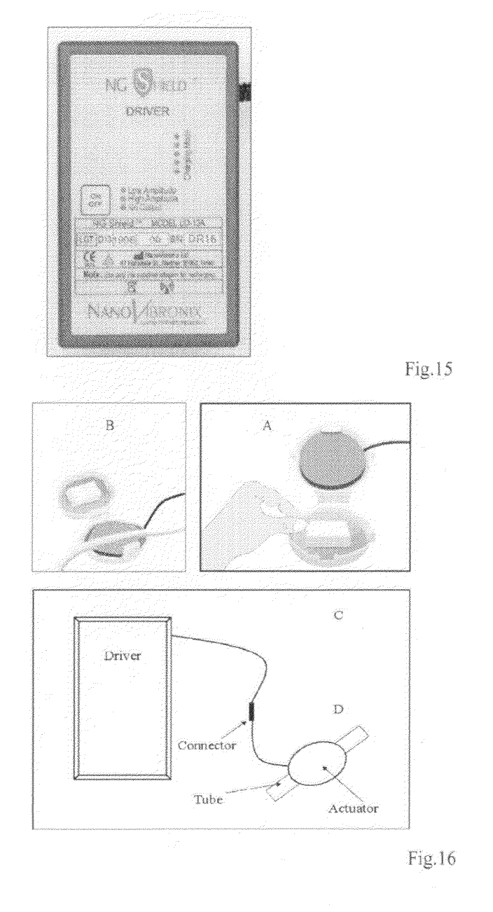

FIG. 14 illustrates an actuator attached to NG tube, while FIG. 15 is an image of an electronic driver unit to be connected to such an actuator in order for the actuator to provide acoustic lubrication effect and/or a rolling effect.

FIG. 16 illustrates acoustic lubrication actuator placement procedure.

In exemplary embodiments of the invention, the actuator is attached to the indwelling medical device, thereby attaching the apparatus of the present invention to the device.

In some embodiments of the invention, the actuator and the driver are incorporated into one case. The connection of the actuator to the driver may be direct or indirect, e.g. through a cable. In one embodiment, an actuator and the driver, incorporated into one case, are attached to an indwelling medical device.

The actuator's initial optimal position is on the distal extracorporeal end (non-invasive end) of a NG tube, such that the medical personnel placing the actuator can feel slight vibrations at the entrance of the NG tube into the nares (the pair of openings of the nose or nasal cavity), but such that the patient feels minimal vibrations or does not feel any vibrations. Subsequently, the actuator may be positioned by sliding it proximally or distally while it is in use. The optimal position is judged on the basis of the patient's nasal and pharyngeal symptoms.

In some embodiments of the invention, the actuator is attached on a non-invasive end of the indwelling medical device.

In other embodiments of the invention, the actuator may be repositioned while in use by sliding the actuator along the indwelling device either proximally or distally to an invasive end of the indwelling medical device.

In an exemplary embodiment of the invention, attachment of the actuator to the tube is via a clip-on stabilization mechanism.

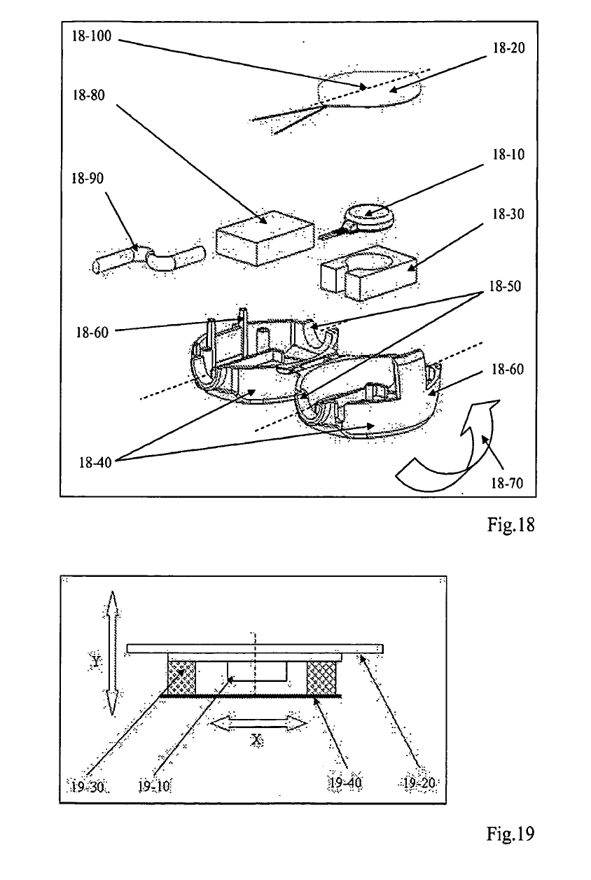

A disposable actuator of the invention is shown in FIG. 18. The disposable actuator is a small and lightweight clip-on actuator that is attached to the tube after the tube is inserted into the patient. In some embodiments of the invention, the actuator is attached to the tube prior to inserting the tube into a patient. In other embodiments of the invention, the actuator is attached to the tube following insertion of the tube into the patient.

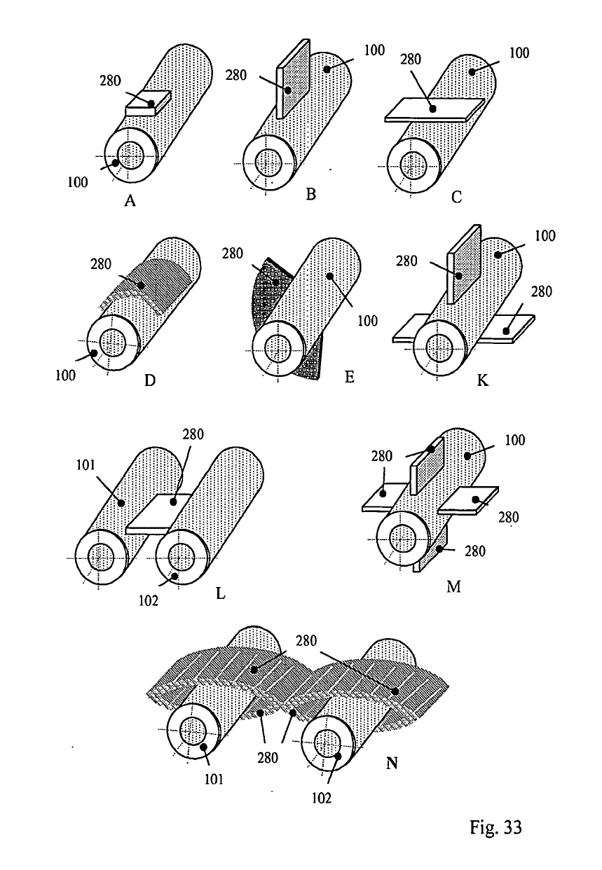

FIGS. 33A, 33B, 33C, 33D, 33E, 33K, 33L, 33M and 33N illustrate different methods for attaching an actuator to an indwelling medical device. The illustrations in FIG. 33 show a thin plate shape piezo ceramic actuator 280 attached to medical tube 100 in several different manners, for example: in parallel to tube surface (33A, 33C), perpendicularly (33B), three actuators perpendicularly (33K), four actuators perpendicularly (33M), as a bridge between two tubes channels (33L).

In one embodiment, the ratio of a medical indwelling device to an actuator is one to one (1:1). In one embodiment, the ratio of a medical indwelling device to an actuator is two to one (2:1). In one embodiment, the ratio of a medical indwelling device to an actuator is two to two (2:2). In one embodiment, the ratio of a medical indwelling device to an actuator is two to four (2:4). In one embodiment, the ratio of a medical indwelling device to an actuator is one to two (1:2). In one embodiment, the ratio of a medical indwelling device to an actuator is one to three (1:3). In one embodiment, the ratio of a medical indwelling device to an actuator is one to four (1:4).

In one embodiment of the invention, a system of the invention comprises one medical indwelling tube. In another embodiment of the invention, a system of the invention comprises more than one medical indwelling tube. In one embodiment, there are two medical indwelling tubes. FIGS. 33L and 33N illustrate different methods for attaching an actuator to an indwelling medical device, wherein the system comprises two indwelling medical tubes 101 and 102.

In other embodiments of the invention, the driver powers the actuator. In one embodiment, the driver powers the actuator, wherein an electrical signal is generated that controls the functionality of the actuator unit and displays the functionality of the actuator unit. In some embodiments of the invention, the driver powers the actuator using batteries contained within the driver. In some embodiments of the invention, the actuator receives electrical energy from the driver and wherein the actuator responds by generating complex vibrations.

In one embodiment or the invention, the acoustic lubrication effect is a result of vibrations from a piezo mechanical vibrator, vibrations from an electro mechanical vibrator, or from combined vibrations from a piezo mechanical vibrator and an electro mechanical vibrator. In exemplary embodiments of the invention, the acoustic lubrication effect is a result of combined vibrations from a piezo mechanical vibrator and an electro mechanical vibrator.

Similar to the description of use of the NG Shield System described in more details below for acoustic lubrication excitement in the NG tube, other medical tubes may be incorporated into such a system creating complex vibrations of acoustic lubrication and rolling effect on their surfaces by applying the same principles.

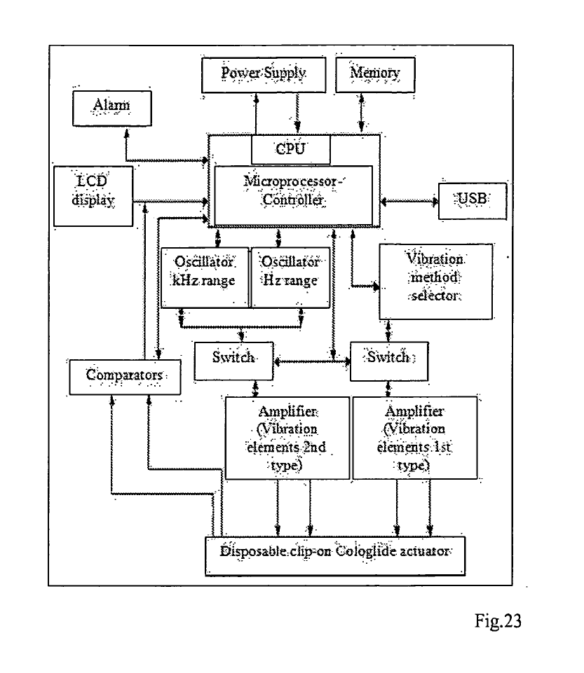

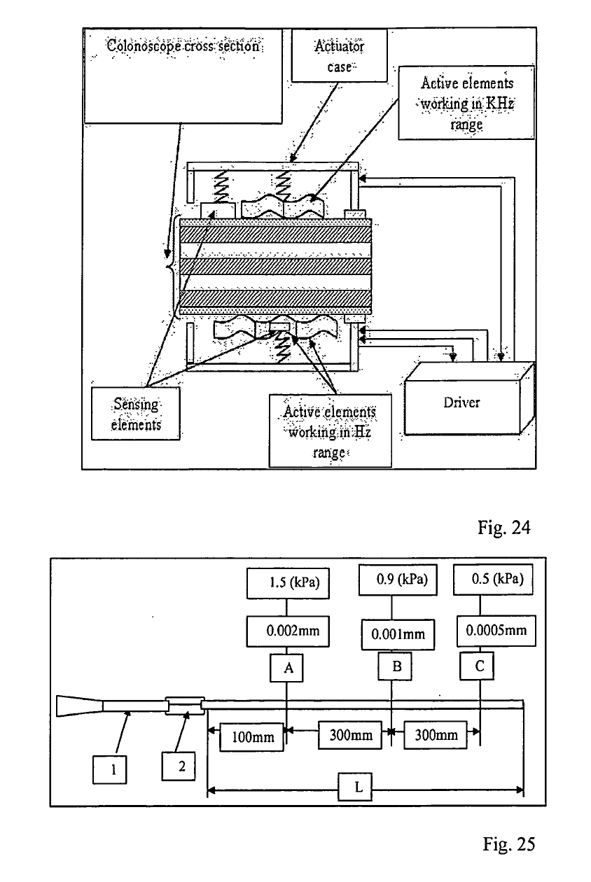

An example of another system may comprise a device for friction reduction between a colonoscope interface and vital tissue, comprising a reusable vibrations actuator and a driver. The principle scheme of a reusable vibration actuator is shown in FIG. 24. The principle scheme of a driver is shown in FIG. 23.

In an exemplary embodiment of the invention, the indwelling medical tube is a colonoscope.

FIG. 4 illustrates acoustic waves traveling through a tube surface, in this instance a catheter, and transferring energy from one point to another with little displacement of the particles of the tube material. These waves produce micro-vibrations on the tube surface.

In one embodiment, this invention provides a system comprising an apparatus as described herein, wherein the apparatus is for generating vibrations along a surface of an indwelling medical device that is in contact with a vital tissue, the vibrations comprising: (a) Hz frequency vibrations; or (b) kHz frequency surface acoustic waves (SAW) vibrations; or (c) a combination thereof, wherein the vibrations create an acoustic lubrication effect along the surface of the indwelling medical device that is in contact with the vital tissue.

In one embodiment of the system, low frequency Hz range vibrations create an acoustic lubrication effect due to actuator's generated cylindrical shape acoustic waves with different wave length propagation on the tube surface through its length.

In one embodiment, the system further comprises an indwelling tube, a display, a computer, a user board, an imaging system, handles, fixtures, stickers, adhesion pads, stands, timers, detectors, sensors, attachments and/or a combination thereof.

In some embodiments of the invention, an apparatus of the invention comprises an electrical energy driver and an actuator, wherein the driver activates a piezo mechanical vibrator within the actuator, wherein the thin plate piezo mechanical actuator is capable to generate complex vibrations comprising low frequency Hz range vibrations and kHz frequency surface acoustic waves (SAW), wherein the apparatus is incorporated into a medical shoe pad such that the active elements may make contact with a subject's foot through a silicone layer of the shoe pad, wherein the contact leads to enhanced tactile sensation in the foot dependent on changes in pressure under the foot. In one embodiment of the invention, an apparatus is for generating micro-vibrations within a medical shoe pad, wherein the vibrations comprise: (a) Hz range vibrations; or (b) kHz range surface acoustic wave (SAW) vibrations; or (c) a combination thereof, wherein the vibrations enhance tactile sensation in a foot dependent on changes in pressure created due to the vibrations.

In some embodiments, the apparatus for generating vibrations within a medical shoe pad comprises an actuator and a driver. In one embodiment of the apparatus for generating vibrations is within a medical shoe pad. In one embodiment, the actuator comprises a piezo mechanical vibrator.

In one embodiment of the invention, an apparatus for generating vibrations within a medical shoe pad, the driver powers the apparatus by activating the actuator. In one embodiment, the driver powers an actuator comprising a piezo mechanical vibrator.

In one embodiment of the invention, the apparatus for generating vibrations within a medical shoe pad is incorporated into the medical shoe pad.

In one embodiment, micro-vibrations are transferred from a piezo mechanical vibrator through a silicone layer of the shoe pad to the patient's foot.

In one embodiment of an apparatus for generating vibrations within a medical shoe pad, the piezo mechanical vibrator creates vibrations at about 90-120 kHz and/or 30-70 Hz.

In one embodiment, vibrating elements may be the thin piezo ceramic plates capable to create surface acoustic waves, such as those described in the examples and United States Patent Application Publication No. 20050268921, Zumeris et al. “Nanovibration coating process for medical devices using multi vibration modes of a thin piezo element”, which is herein incorporated by reference.

FIG. 32 present three examples of micro massage foot pads, which incorporate an apparatus of this invention, wherein vibrations may be generated to enhance tactile sensation in a foot depending on changes in pressure created due to the vibrations.

### IV. METHODS OF OPERATION

In some embodiments of the invention, an apparatus generates vibrations that propagate on the medical tube surface. As a result the contact time and contact area between the two surfaces (tube and vital tissues) is reduced.

In one embodiment, the at least one vibration element of an actuator generates mechanical self vibrations in the Hz range with maximum amplitudes reaching about 100 microns. “Self” or “natural” or “free” vibration occurs when a mechanical system is set off with an initial input and then allowed to vibrate freely. In some embodiments, the amplitude of Hz range self vibrations of the vibration element ranges between 10 microns and 100 microns, wherein amplitude refers to amplitude of the vibration element oscillations and not the amplitudes along the surface of the tube created due to vibration element oscillations. In other embodiments, the amplitude of kHz range vibrator's self vibrations amplitudes reaching 20 micron.

Thus, in one embodiment of the invention, when the frequency of acoustic lubrication created on the medical tube surface is in the range between 30 and 200 Hz, the resultant medical tube surface maximal displacement (or surface vibration amplitudes) is about 1-10 micron (1 micron=0.001 mm) at a distance of 10 cm from the vibrator The attached actuator touches the tube surface and creates vibrations on the medical tube surface. These vibrations are so called acoustic lubrication. Surface max displacement is equal to max surface vibration amplitude.

As used herein, the term “vibrator” may also be referred to herein as an “actuator” or a “vibrator element”, have all the qualities and properties of a vibrator.

In some embodiments of the invention, when the frequency of acoustic lubrication created on the medical tube surface is in the range between 90 and 120 kHz, the resultant medical tube surface maximal displacement (surface vibration amplitude) is about 0.1-1 micron.

In one embodiment, the vibrations generated comprise vibrations of about 30 Hz, of about 100 Hz and of about 100 kHz.

Reference is now made to FIG. 10, illustrating a combination of low frequency acoustic waves propagating on a medical devices surface, wherein: 11-10 is an indwelling medical device, 11-20 is an actuator comprising Hz range and kHz range frequency vibration elements. 11-30 is a driver, 11-40 shows an acoustic waves propagation process, and 11-50 shows mathematical simulation of device diameter changes due to oscillations.

Laboratory measurements have shown that combined Hz range vibrations creating acoustic lubrication and kHz range vibrations creating acoustic rolling effect reduces the friction coefficient considerably on both dry and wet medical tube surfaces. The “wet” tube may be considered as a tube covered with lubricating jelly (see for example measurement results in FIG. 27).

In some embodiments of the invention, low frequency acoustic waves generate pressure along the medical indwelling device.

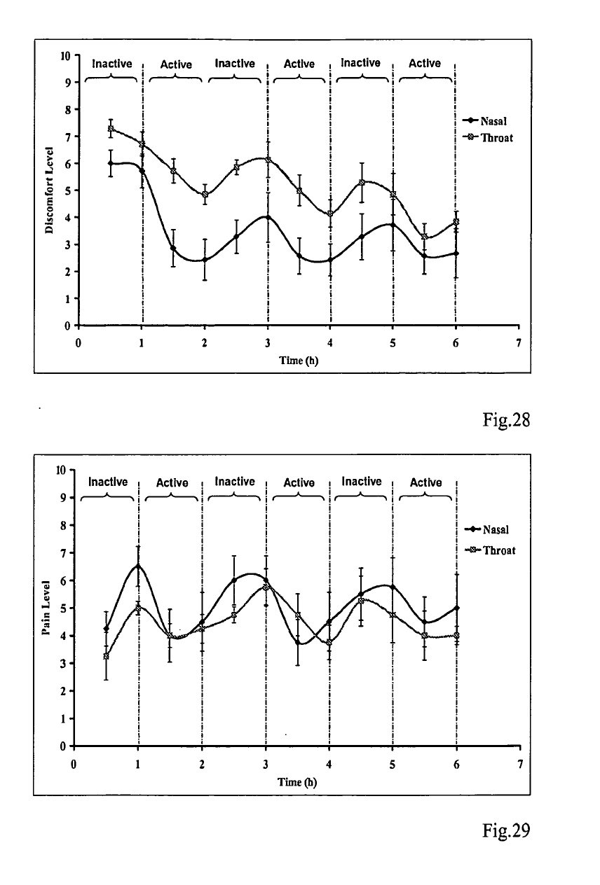

The maximal pressure created due to the acoustic lubrication effect is safe to the user. FIGS. 25 and 27 present results of the measurements and calculations that underscore the safety of acoustic lubrication. Clinical tests with healthy volunteers, provided in FIG. 28 and FIG. 29, demonstrated that acoustic lubrication markedly decreased pain and discomfort associated with NG tube use.

In one embodiment of the invention comprising a driver and an actuator that provide vibrations for the reduction of friction between an indwelling medical tube and a vital tissue, when the driver is turned on, the actuator mechanically vibrates and generates cylindrical type Hz range acoustic waves on the NG tube surface with maximal displacement of +/−1 micron at 10 cm distance from the actuator, which are gradually damped such that there are no vibrations at the far end of nasal gastric tube. These acoustic waves reduce the contact time between the tube and the naso-pharyngeal tissues. The effective friction between the tube and patient's nasopharyngeal tissue is minimized, thus lessening the damage that might be caused to the mucosa of nasopharyngeal tissues in contact with the tube.

In some embodiments of the invention, kHz frequency SAW create “rolling” type tube surface particle motion. The waves have frequencies ranging between 90-120 kHz. In exemplary embodiments of the invention, kHz range frequency SAW “rolling” type vibrations have an amplitude of +/−1 micron at 10 cm distance from the actuator (1 micron is equal 0.001 millimeter).

The acoustic lubrication effect is enhanced due to an additional element of the actuator, a PZT element, which is a piezo mechanical vibrator which creates surface acoustic waves on the tube surface. These waves generate circular material particles movements, thus creating rolling effect in the interface points. Rolling effect decreases the friction and in the combination with Hz range vibrations results in effective acoustic lubrication and therefore reduces patient's pain.

In some embodiments of the invention, the actuator comprises at least two vibration elements, a piezo mechanical vibrator (frequency range 90-120 kHz and/or 30-70 Hz) and an electromechanical vibrator (frequency range 70-200 Hz).

Electromechanical vibrators (i.e., miniature electromechanical motors which are suitable for current invention) are generally unable to create vibrations with a frequency lower than about 100 Hz. This is in comparison to piezo electric vibrator, which is capable of creating vibrations with a frequencies as low as 30 Hz and even lower. It is well known that lower frequency waves are less attenuated and therefore such low frequency waves may better reach the end of a long tube, such as colonoscope

In order to create lower Hz range vibrations, for example 30 Hz vibrations, which due to longer wave length and lesser attenuation can propagate further along a long tube to the tube's end, the same piezo mechanical vibrator may be used to generate lower Hz range vibrations and higher kHz range vibrations.

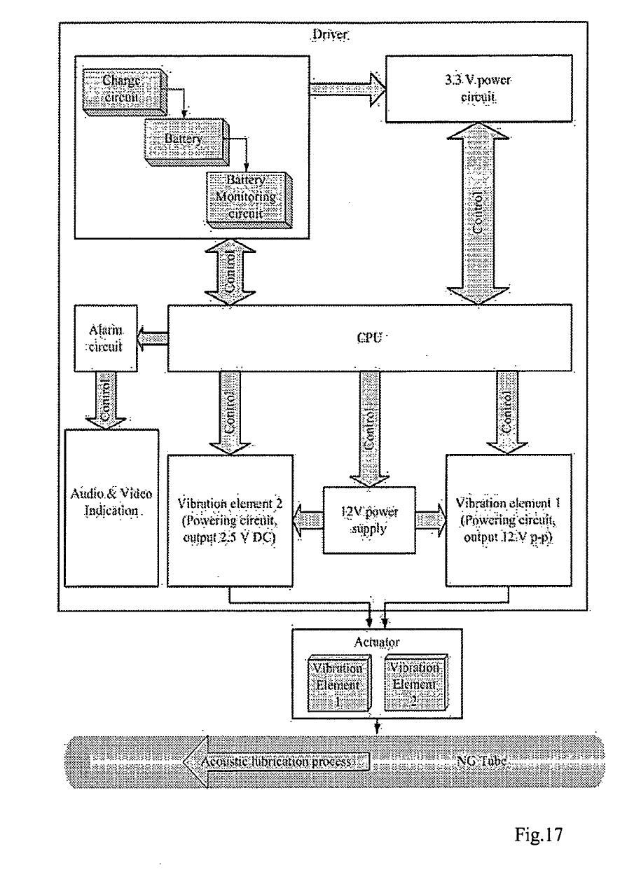

A schematic illustration of an exemplary embodiment of the invention apparatus is shown in FIG. 17, illustrating the connection and relationship between elements of the apparatus and their function in the creation of the acoustic lubrication process that is inclusive of low Hz range and kHz freq. waves. The driver contains the Central Processor Unit (CPU), rechargeable batteries to power the system and the electronic circuit to drive the actuator. The rechargeable lithium battery powers the driver unit with 7.4V DC. The output of the battery is converted into 2 separate DC signals: a 12V signal and a 3.3V signal. The 12V signal is used to produce a 2.5V DC signal to activate the vibration element 1 which is based on electro mechanical principles and creates low frequency Hz range vibrations which are acoustic lubrication source. In addition this signal power vibration element 2 which is based on piezo mechanical principles and may create low frequency Hz range vibrations which are acoustic lubrication source and/or kHz range surface acoustic waves, which are the rolling effect source. The 3.3V DC signal is used to power the CPU. The CPU, in turn, controls the driver. Its functions include: determining the frequency for vibration element and monitoring battery power and activating a buzzer alarm when the voltage level is low. Driver specifications: DC to DC converter (7.4V DC to 12V DC); Output signal to vibration element 1 (2.5V DC); Output signal to vibration element 2 (12V p-p with 100 kHz signal modulated with 30 Hz off/on); Total current consumption for both vibration elements 250 mA.

In an exemplary embodiment of the invention, the piezo mechanical vibrator creates vibrations at about 100 kHz.

In an exemplary embodiment of the invention, electro mechanical vibrator creates vibrations at about 70-200 Hz range.

In an exemplary embodiment of the invention, the piezo mechanical vibrator creates vibrations at about 30 Hz.

Technical characteristics of an apparatus creating acoustic lubrication on colonoscope include: an actuator comprising a small and thin electro mechanical vibrator; with a vibrator self vibration amplitude range of 1 to 100 microns; a controlled energy transmission due to a close loop algorithm; and a programmable energy level and treatment time. The acoustic lubrication reduces friction between a medical indwelling device (e.g., a colonoscope) and a vital tissue, and therefore contributes to decreasing pain in a subject. Self vibrations comprise vibrations generated on the actuator's surface, which have a higher amplitude ranging in about 100 micron. When the actuator is attached to a medical tube, e.g. a colonoscope, vibrations are created along the colonoscope surface; the vibrations along the tube surface are in the 0.1-10 micron range. It is the vibrations along the tube surface that result in acoustic lubrication.

In addition SAW created with piezo mechanical vibrator, comprised in an actuator, reduce bacterial adhesion in the inner channel of the colonoscope, as it was detailed in United States Patent Application Publication No. 20050268921, thus inhibiting biofilm formation and enhancing sterilization procedures.

In an exemplary embodiment of the invention, the acoustic lubrication effect reduces pain and/or discomfort of a subject. In some embodiments of the invention, pain and/or discomfort are reduced during a limited invasive medical procedure experienced by the subject, involving: insertion, removal, indwelling or any combination thereof, of an indwelling medical device.

In some embodiments of the invention, the complex vibrations reduce bacterial adhesion on an inner channel of an indwelling medical device. In one embodiment, Hz range vibrations or kHz range surface acoustic waves (SAW) or a combination thereof, reduce bacterial adhesion on an inner channel of an indwelling medical device.

The main goals of the technology when it is applied for colonoscopy is that use of an apparatus of this invention leads to the decreased friction (dynamic and static) due to generation of an acoustic lubrication effect; decreased patient discomfort and pain level; decreased use of sedations, less wear and tear on physicians doing repeated procedures; decreased possibility of channel clogging, in addition to reduced bacterial adhesion on an inner channel.

In one embodiment, Hz range vibrations or kHz range surface acoustic waves (SAW) or a combination thereof provide a means for reduced procedural time for minimally invasive medical procedures comprising insertion, removal, indwelling or any combination thereof of the indwelling medical device.

In one embodiment, Hz range vibrations or kHz range surface acoustic waves (SAW) or a combination thereof provide improved ergonomics of indwelling medical devices, thereby increasing efficiency and/or safety of use of the indwelling medical device.

The acoustic vibrations of the system described do not interfere with video signals output and impacts in improved ergonomics: the scope may be easier for navigation and the procedure may be easier for the physician. Another potential benefit is due to decreased friction between tissue and colonoscope, less sedation and/or time will be to caecal intubation (“intubation” or “caecal intubation” equals colonoscope insertion).

In some embodiments of the invention, complex vibration improve definition of structures imaged using an ultrasound scope. In other embodiments of the invention, complex vibrations decrease impedance to electro cautery in a vital tissue. In one embodiment, Hz range vibrations or kHz range surface acoustic waves (SAW) or a combination thereof decrease impedance to electro cautery in a vital tissue. In one embodiment, Hz range vibrations or kHz range surface acoustic waves (SAW) or a combination thereof improve definition of structures imaged using the indwelling medical device.

Similarly, complex vibrations created by embodiments of this invention may improve the quality of action of an implement that inserts into an inner channel of a tube. Embodiments of this invention comprising indwelling medical devices which may have an implement inserted into an inner channel comprise: a nasal-gastric (NG) tube, a colonoscope, a gastroscope, a duodenscope, a bronchoscope, a cytoscope, a cystoscope, a urethroscope, a hemorrhoids treatment tube, a vaginal tube, an ultrasound scope, a catheter, a cauterizing tube, a cannula, a flexible endoscope or any other medical device utilized during minimally invasive procedures.

In some embodiments of the invention, complex vibrations improve a quality of action of an implement that inserts into an inner channel of the medical indwelling device. In one embodiment, Hz range vibrations or kHz range surface acoustic waves (SAW) or a combination thereof improve a quality of action of an implement that inserts into an inner channel of the medical indwelling device.

In some embodiments of the invention, the implement is disposable.

In some embodiments of the invention, the implement comprises a polypectomy snare, a sphincterotome, a papillotome, a needle knife papillotome, or any other implement used for a trans-endoscopic electro surgery. In some embodiments of the invention, the trans-endoscopic electro surgery quality is improved, wherein the improvement leads to minimal injury of tissue.

It is known that use of kHz range frequency improves definition of structures when an ultrasound scope is used.

Embodiments of the invention include applying vibrations directly or indirectly to implements, wherein the implements are disposable or non-disposable, and further, wherein the implement goes into an inner channel of a tube. Such implements include polypectomy snares, sphincterotomes, papillotomes and needle knife papillotomes, as well as other devices used for trans-endoscopic electrosurgery. A polypectomy snare is a wire loop device designed to slip over a polyp and, upon closure, results in transaction of the polyp stalk. A sphincterotome is an instrument for incising a sphincter (a muscle that normally maintains constriction of a natural body passage). In one embodiment, a sphincterotome is used for incising a sphincter. In one embodiment, a sphincterotome is used for cannulation of a ductal system. Papillotome is an electrosurgical endoscopic wire guided catheter that is used in conjunction with a flexible endoscope to diagnose disease, clear obstructions, and restore patency in biliary tract.

FIG. 22 shows such an implement inserted into a colonoscope inner channel and used for electro surgery.

### The NG Shield Apparatus and System

In one embodiment, this invention provides an NG shield apparatus, as described herein. In one embodiment, this invention provides an NG shield system thereof, to as described herein. In some embodiments, the NG shield apparatus provides only low frequency Hz range vibrations. In another embodiment, the NG shield system provides a combination of Hz range and SAW (kHz range vibrations) Example 2 exemplifies some embodiments of an NG shield apparatus and system, and methods of use thereof.

The illustration in FIG. 11 shows an exemplary embodiment of the NG shield apparatus and system thereof, consisting of an actuator (1) connected via a cable (2) to a driver (3), wherein the actuator is attached to the NG tube (4) which is inserted into the patient's nasogastric region. The contact area between the indwelling medical device and the vital tissues during the period of tube usage is marked by arrows (5).

The acoustic wave propagation on the NG tube surface results in a decrease of the real contact area and contact time between the tube surface and the patient's nasopharyngeal tissue that is in contact with the tube. As is common to all acoustic waves, they are gradually dampened. Therefore the above explained cross sectional changes lessen with increasing distance from the actuator.

In an exemplary embodiment of the invention, the indwelling medical device is a NG tube. In such an embodiment the system is known as an NG Shield.

An NG Shield will significantly improve the care of patients with an indwelling NG tubes. In addition, an NG shield system of this invention may prevent or minimize some of the traumatic complications associated with NG tube usage, which at times can result in long standing or permanent complications to patients and therefore additional costs.

As an example of the invention, the acoustic wave(s) generated by an NG shield system are vibrations that travel on the surface of the NG tube material. The NG tube material upon which the wave(s) travels experiences local oscillations as the wave passes, but the tube does not travel with the wave. FIG. 10 illustrates a mathematical simulation of cylindrical type of acoustic waves' propagation along the NG tube. The particle motions are magnified 100 times.

FIG. 17 illustrates in schematic form an apparatus and system thereof comprising an NG tube, wherein the apparatus comprises an actuator with two vibration elements and a driver.

FIG. 18 illustrates an exemplary NG actuator wherein: 18-10—first vibration element; 18-20—second vibration element; 18-30-foam with special hole for first vibration element; 18-40—actuator's case; 18-50—cylindrical guide; 18-60—fixation mechanism; 18-70—clip on direction; 18-80—preload foam; 18-90—cable; 18-100—active contact line between actuator and tube, which creates acoustic lubrication on the NG tube surface.

In an exemplary embodiment, the actuator's vibration mechanism contains two vibration elements: vibration element 18-10 and vibration element 18-20 (elements 1st and 2nd of FIG. 18). These are rigidly fixed together and placed into special shaped hole in the foam element 18-30, which in turn is fixed to the actuator's case element 18-40. The actuator's case element 18-40 consists of two parts and has cylindrical guide element 18-50 for NG tube fixation. When the NG tube is fixed in the cylindrical guides the actuator's case is closed in the direction 18-70 using the clip-on mechanism element 18-60. Foam element 18-80 enables proper preload of the NG tube to the vibration mechanism in the area of the active contact line 18-100. Vibration elements 18-10 and 18-20 are electrically connected with the driver through cable 18-90.

A schematic representation of the actuator's vibration mechanism is shown in FIG. 19: 19-10—first vibration element; 19-20—second vibration element; 19-30—foam with special shaped hole for vibration element 1; 19-40—actuator's case; X—direction of the vibrations created by vibration element 19-10; Y—direction of the vibrations created by vibration element 19-20. Vibration element 19-10 is an electro mechanical 12 mm shaftless vibration motor, button type model 312-103, manufactured by Precision Microdrives. It is powered by a 2.5 V DC volt signal, and creates horizontal (X direction) vibrations. The frequency of these vibrations is about 100 Hz+1-3 Hz. In one embodiment, an electromechanical vibration element is referred to as an electromechanical vibrator. Vibration element 19-20 creates vibrations in the Y direction (perpendicular to the tube surface). Element 19-20 is a piezo electric mechanical vibrator, manufactured by American Piezo Ceramics, powered by a 12V p-p AC current signal, creating the main vibration frequency of 30 Hz+/−1 Hz. In order to create 30 Hz vibrations using piezo mechanical vibrator, the vibrator is excited to vibrate in kHz range and these vibrations are turned on/off in 30 Hz frequency. All this is necessary, because 30 Hz range could not be achieved with electromechanical vibrators—such electromechanical vibrators do not exist. In one embodiment, the term “piezoelectric actuator element” is referred to as a piezo mechanical vibrator.

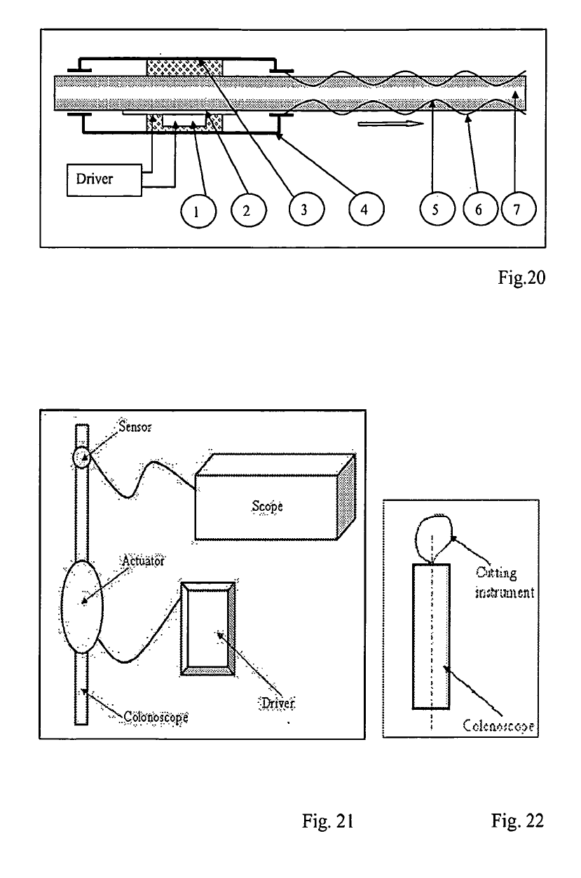

Reference is now made to FIG. 20, which schematically illustrates the NG actuator attached to NG tube and acoustic wave propagation along the NG tube surfaces, resulting in the generation of the acoustic lubrication effects wherein: 20-1—is a first vibrating element and it's vibrations direction—as described in FIG. 19; 20-2—is a second vibrating element and it's vibration direction, as described in FIG. 19; 20-3—foam with special shaped hole for vibrating element 1 and for vibrating element preload; 20-4—actuator case; 20-5 and 20-6—acoustic waves propagations; 20-7—NG tube for acoustic lubrication between tissues and NG tube. NG Shield actuator specifications: Frequency of vibration element 1 (100+/−3 Hz); Frequency of vibration element 2 (30+/−1 Hz).

In an exemplary embodiment of the invention, one vibration element of an actuator vibrates with a frequency of vibration at 100+/−3 Hz and another vibration element of the same actuator vibrates with a frequency of vibration at 30+/−1 Hz.

The sources of pain in patients with an indwelling NG tube are in the nasal and/or pharyngeal regions. Keeping this in mind and keeping in mind that wave length is dependant on frequency, we designed theoretically and following have chosen experimentally the optimal regimen of actuator activation which was 30 Hz and 100 Hz simultaneously. The wave length corresponding to 30 Hz is longer than generated with 100 Hz. These vibration modes within the actuator are created with two vibration elements, as described above.

Low frequency vibrations of 70-200 Hz may be achieved with the use of electromechanical motor. While the longer wave length, which may be achieved with lower frequency ranges, for example with 30 Hz vibrations, could not be achieved with electromechanical motor, because there are no such electromechanical motors. In one embodiment, the purpose is to add 30 Hz frequency to the combination of vibration frequencies. This can be achieved as follows: The frequency of 30 Hz vibration is achieved with vibration element 2 (piezo element) in two phases: element 2 is excited to vibrate at its natural vibration frequency which is about 100 kHz, and these vibrations are modulated with 30 Hz in an on/off regimen. The combined vibrations (100 Hz of electromechanical vibrator and 30 Hz and 100 kHz with piezo element 2) have a summary of frequencies that act as acoustic lubrication and provide the desired results by means of decreasing the real surface of contact between the patient's nasopharyngeal tissues and the tubes outer surface. The following characteristics were shown experimentally in the examples below: the combination of vibrations consisting of 100 Hz (created with vibration element 1) together with 30 Hz and 100 kHz (created with vibration element 2) have shown a reduction in the effective friction coefficient by more than 20% (as shown in the report in FIG. 27; see overall Change in Coefficient of Friction).

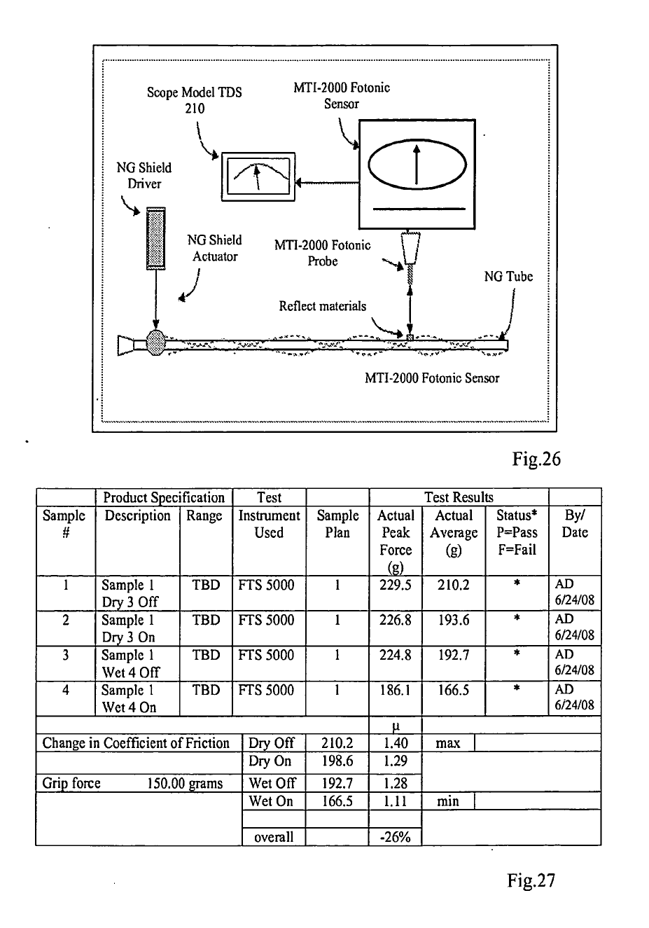

FIG. 26 details the set up for measurements of the displacement amplitudes and frequency on the NG tube (created with the vibration actuator attached to the NG tube) using a MTI-2000 Fotonic Sensor. Calculations using these experimental results show the maximal pressure on the NG tube system to be equal to 800 Pa. Furthermore, the results of in vivo animal studies pointed out that the device is safe. The histopathological analysis performed during this study showed less tissue damage in the tract of animals that were treated with the active device compared to the animals from the control group (data not shown).

In summary, the acoustic lubrication effect of the device may be considered as if the surface of the tube is constantly lubricated through the duration of its usage. The effect is advantageous because its action is non-permanent, and is continuous during the use of the NG tube. This is contrary to lubricating jelly which creates lubrication only at the beginning of NG tube usage. Moreover, as was demonstrated in the clinical trial with healthy volunteers in Example 4, the patients felt less pain and discomfort when acoustic lubrication was turned on, as compared to the periods when the acoustic lubrication was turned off.

### A Cologlide Apparatus and System

Colonoscopy is the leading, most frequently used procedure for detection, diagnosis and removal of benign and malignant polyps in the colon. The annual number of procedures is growing. The continuous growth in the number of required colonoscopies, leads to market opportunities for methods and techniques that: improve the colonoscope movement & maneuvers; reduce procedure's duration; reduce pain, level of patient sedation and recovery time; reduce rate of complications

Current limitations in colonoscopy are that the lubricant gel used for insertion is effective for anus region only, sedation to minimize pain requires ˜2 hours for recovery, and colonoscope cleaning is done with washing machines which do not fully prevent the risk of contamination.

In one embodiment of the invention, a colonoscopy system provides acoustic lubrication by reduction of tube-tissue contact time to −50%; decreases the risk of colonoscope looping and pain resulting from distention of the colon and its mesentery, and provide acoustic waves to tube inner lumen to prevent bacterial adhesion that is followed by biofilm formation (colonization).

It is envisioned that use of this invention may lead to development of algorithms for optimal modulation of waves to: e.g., vibrate the scope, throughout its tip, to minimize tube-tissue contact time; decrease the risk of colonoscope looping and pain resulting from distention of the colon and its mesentery; provide acoustic energy in the form of SAW to the inner lumen for prevention of bacterial adhesion and/or any combination of these without interfering with a camera video signal and other working modules.

An apparatus of this invention will generate and propagate low frequency, low intensity acoustic waves on colonoscope surfaces. Creating these low frequency, low intensity acoustic waves may benefit the easy fitting to any standard colonoscope while not interfering with optical or ultrasound imaging signals. Optional therapeutic advantages include easier polyps suction.

Similar to the actuator described as part of the NG shield system, active vibration elements of an activator for use with a colonoscope may create a combination of vibrations of multimode wave propagation, including cylindrical waves and SAW type.

Mathematical simulations of cylindrical type traveling acoustic waves show that such waves:

1. Vibrate the colonoscope scope through its entire length to reduce tube-tissue contact time (acoustic lubrication);

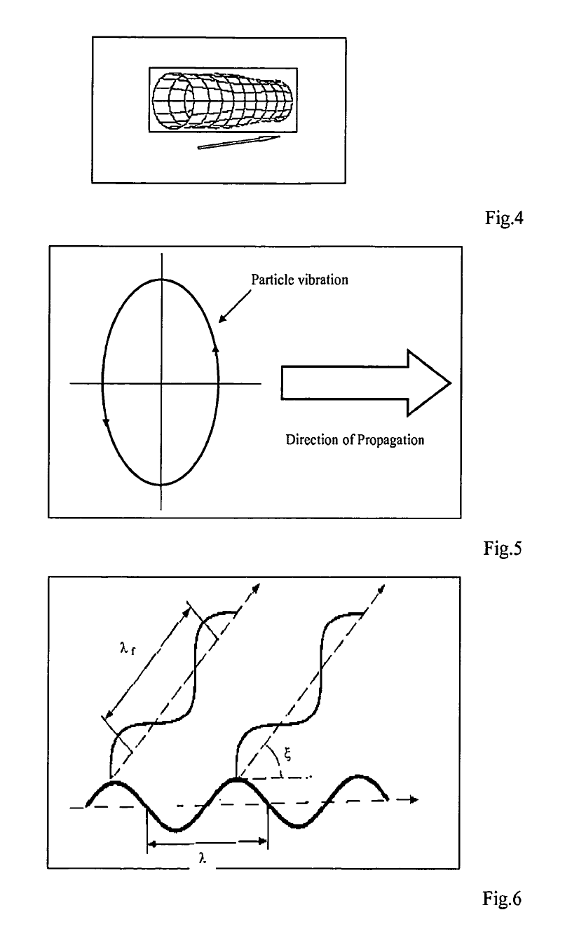

2. Produce micro massage effect in tissue and therefore effect pain reduction. See for example, FIG. 6, which shows the generation of compression waves into tissue (λ1) by a SAW (λ) wave motion on the surface at an angle (ξ);

3. Propagate SAW on a tube outer surface and inner lumen surfaces resulting in rolling effect which reduces friction. In addition, SAW (Rayleigh wave) motion on the surface of medical device inhibit bacterial adhesion due to relative velocity of bacteria respectively to vibrating surface, as it was explained in details in United States Patent Application Publication Number 2007/0213645, by Zumeris et al. which is fully incorporated herein by reference.

Benefits of an apparatus and system thereof, designed around a colonoscope indwelling device include reduced tube-tissue contact time and friction lead to easier scope insertion, movement and maneuvers; increasing the procedural convenience for the physician; reducing patient pain and/or discomfort; reducing duration of a procedure and the rate of complications (perforation). Further, reduction of patient suffering allows reduction in sedation level leading to better communication between the physician and the patient, shorter recovery time periods.

Use of a colonoscope often leads to development of a biofilm in the functional inner channel of the colonoscope, through which drugs or suction means are inserted. Biofilm cleaning during colonoscope disinfection processes requires an amount of chemicals which damages the surface of the indwelling device and often is not fully effective. In one embodiment of the invention, an apparatus of the invention prevents development of a biofilm in the inner channel of a tube. Biofilin prevention reduces colonoscope contamination. In one embodiment, an apparatus of the invention reduces contamination of an inner channel of an indwelling medical device. In one embodiment, an apparatus of the invention reduces colonoscope inner channel contamination.

In addition, vibrations on the medical tube surface will decrease impedance to electro cautery in tissue when surface vibrations are applied to the scope. The polyp suction procedure then may become easier to manage for physicians due to reduced friction between tissue and a cutting element.

FIG. 23 shows a schematic for an acoustic lubrication system for a colonoscope. FIG. 24 shows a schematic for a disposable clipped on actuator and associated driver, for acoustic lubrication with a colonoscope.

### V. METHODS OF USE AND APPLICATIONS

There is a clear need, expressed by physicians and patients, for apparatus and systems thereof, of the current invention and uses thereof, wherein in some embodiments use of a system of the invention is capable of alleviating the detrimental effects associated with indwelling medical devices and improving ease and safety of such use.

In one embodiment of this invention, a method for generating an acoustic lubrication effect along a surface of an indwelling medical device that is in contact with a vital tissue in a subject, comprises use of an apparatus as described herein, for generating: (a) Hz range vibrations; or (b) kHz range surface acoustic wave (SAW) vibrations; or (c) a combination thereof, wherein the vibrations create an acoustic lubrication effect along the surface of the indwelling medical device that is in contact with the vital tissue. In one embodiment, the method comprises use of a system as described herein.

A NasoGastric tube is a flexible plastic/rubber tube that is inserted through the nose, down the back of the throat, through the esophagus and into the stomach. The NG tube has become one of the most frequently used devices in hospitals. It is also considered to be one of the most painful and/or uncomfortable devices and can result in temporary or permanent tissue damage. Increased pressure and friction between the tube and the tissue (during insertion or while in use) are among the major factors contributing to the pain, discomfort and potential tissue damage.

Colonoscopy is an endoscopic medical procedure where the colon of the patient is examined. This minimally invasive technique is performed with a colonoscope, a long tube with an integrated imaging device at its tip. The doctors performing these procedures require high skills in multiple domains such as hand-eye coordination, visualization, safety and ease at guiding flexible endoscopes. The importance of training colonoscopy procedures rises with the growth of variety of colon diseases.

The present invention includes apparatus and systems thereof and methods of use thereof, for decreasing the pain and discomfort commonly associated with minimally invasive procedures, for example nasal gastric tube insertion and endoscopic procedures, where such procedures may be performed with lower dosage levels of sedative and analgesic drugs.

In some embodiments of the invention, methods comprise generating complex vibrations at an interface between an indwelling medical device and a vital tissue of a subject that the indwelling medical device contacts, wherein the complex vibrations comprise low frequency (Hz range) acoustic lubrication vibrations and surface acoustic waves (kHz range) creating rolling effect, and wherein the low frequency cylindrical type vibrations create an acoustic lubrication effect and the SAW create rolling effect, and wherein the complex vibrations reduce friction at an interface between the indwelling medical device and the vital tissue.

In exemplary embodiments of the invention, method comprises use of an apparatus as described herein and a system thereof described herein, for generating complex vibrations along a surface of an indwelling medical device wherein the complex vibrations reduce friction at an interface between the indwelling medical device and a vital tissue of a subject that the indwelling medical device contacts, wherein the complex vibrations comprise low frequency (Hz range) cylindrical type vibrations and surface acoustic waves (kHz range), and wherein this complex creates an acoustic lubrication effect with rolling effect

In some embodiments of the invention, methods producing complex vibrations reduce pain and/or discomfort in a subject.

In some embodiments of the invention, methods include apparatus and systems thereof, wherein the indwelling medical device comprises a nasal-gastric (NG) tube, a colonoscope, a gastroscope, a duodenscope, a bronchoscope, a cytoscope, a cystoscope, a urethroscope, a hemorrhoids treatment tube, a vaginal tube, an ultrasound scope, a catheter, a cauterizing tube, a cannula, a flexible endoscope or any other medical device utilized during minimally invasive procedures.

In some embodiments of the invention, methods are directed to a subject undergoing a minimally invasive medical technique comprising insertion, removal, indwelling or any combination thereof of an indwelling medical device.

In some embodiments of the invention, methods comprise a minimally invasive medical technique comprising naso-gastric tube insertion; an endoscopic procedure comprising a colonoscopy, imaging technique, hemorrhoid removal, trans-endoscopic electro surgery or any combination thereof; frigidity treatment or any combination thereof.

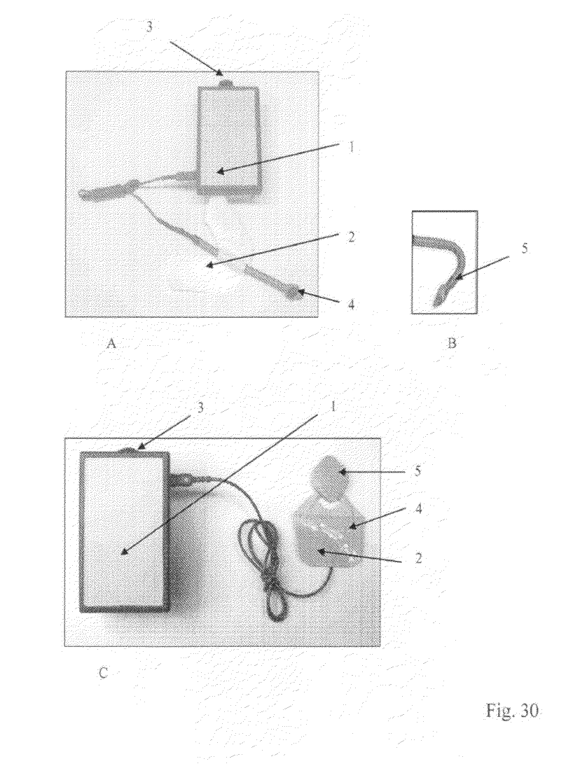

FIG. 30 presents a system for use with frigidity treatments. In one embodiment, a method of use comprises a patch based system for frigidity treatment in women and for enhancing sexual intercourse. FIG. 30 illustrates the device which consists of two parts: a portable electronic driver (1) and a disposable application patch (2), which is connected to the electronic driver. The device may be turned on or off with a relay (3) which allows applying and controlling energy level and therefore creating more or less vibrations, as it will best fit to each patient. The disposable patch should be placed adjacent to the sensitive area of clitoris, following the device turning on. In order to reach an effective touch and preload between vibrating element and skin, the woman should ware the trousers. It is proposed to use the patch for about 15-30 min. in order primarily enhancing sexual intercourse.

Two versions of the patch are shown. FIG. 30 A and FIG. 30 B show a miniature motor based (electro mechanical vibrator—active element 5) patch designed to gently mechanically stimulate the clitoris and to achieve increased blood flow in this area, in “hands free” configuration. The miniature vibration element inserted into the patch end may be placed near the clitoris due to element (4) creating flexibility, incorporated into the patch, as it is shown in FIG. 30 B.

FIG. 30 C shows a novel piezo mechanical element based patch, designed to achieve increased blood flow in this area due to micro massaging and surface acoustic waves (SAW) action. The patch is designed for “hands free” configuration. The active element (5) on the patch end should be placed in the proximity to the sensitive area such that the metallic surface of the active element (5) will be in touch with the skin. FIG. 30 C: patch based device consisting of electronic driver (1) with push button on/off (3) and disposable patch (2) with SAW applicator (5) at the end. The disposable patch has a flexible element (4).

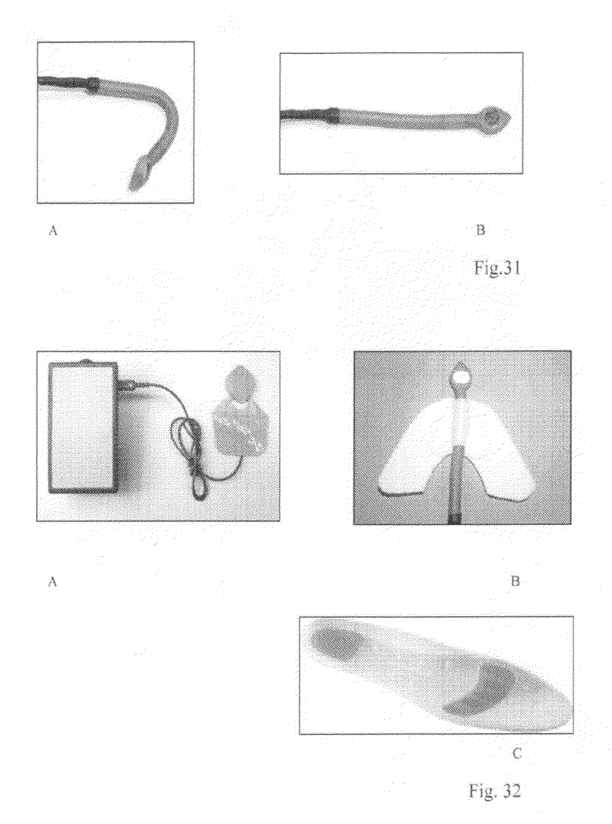

FIG. 31 shows a system for use for hemorrhoid treatments consisting of a flexible tube (see view A and B) containing a small vibration element at the indwelling end of it, which creates acoustic lubrication effect therefore reduce pain at hemorrhoid site.

### EXAMPLES

### Example 1

### Creation of an Acoustic Lubrication Effect on the Entire Length of Catheter Surface

The apparatus created an acoustic lubrication effect over the entire length of the catheter surface. The pressure, frequency and amplitudes of these vibrations were measured in the Nanovibronix Ltd. Acoustic laboratory (using an MTI-2000 Fotonic Sensor). The results showed that vibration amplitudes created on nasal-gastric catheters with acoustic lubrication apparatus attached were in the range of 0.001-0.00025 mm., wherein the complex of vibration frequencies consist of 30 Hz, 100 Hz and 100 kHz, and the measurements were done at a distance between 10-70 cm from the actuator (see FIG. 26).

The study was conducted in acoustic laboratory conducting measurements with MTI-2000 Fotonic Sensor. While working under special operating conditions as per calibration manual for MTI-2000 Fotonic Sensor the digital display was operated in the volts mode and manually calibration of the probe gap vs. output signal were done. Therefore the analog displacement signal (voltage signal) was converted to engineering units (mm).

These vibrations created pressure which was proved to be safe. The animal safety trial showed that this device was absolutely safe.

### Example 2

### Vibration Amplitude Measurements and Maximal Pressure Calculations on a NG Tube with a NG shield Actuator

The purpose was to calculate the pressure of acoustic lubrication created with NG Shield apparatus, also known as an NG Shield device, on nasal-gastric tissue. The value of the pressure contains vibration amplitude and vibration frequency values, therefore if amplitude and frequency were measured, the pressure value could be calculated.

### Experimental Procedure

Lubrication vibrations on the tube surface create vibration pressure. The SI unit for sound pressure is the Pa. The instantaneous vibration pressure is the deviation from the local ambient pressure (p) caused by a vibration wave at a given location and given instant in time. In a vibration wave, the complementary variable to vibration pressure is the acoustic particle velocity. For small amplitudes, vibration pressure and particle velocity are linearly related and their ratio is the acoustic impedance. The acoustic impedance depends on both the characteristics of the wave and the medium. The local instantaneous vibration intensity is the product of the vibration pressure and the acoustic particle velocity and is, therefore, a vector quantity.

The sound pressure deviation p is expressed in pascals (Pa). Vibration pressure p may be described by the equation:

p=ρcωξ=Zωξ=2πfξZ

wherein:
p—sound pressure (Pa)
p—density of air (kg/m³)
c—speed of sound (m/s)
ω=2·π·f—angular frequency (radians/s)
f—particle vibration frequency (Hz)
v—particle velocity (m/s)
ξ—particle displacement (m)
Z=c·p—acoustic impedance (N·s/m³)

Particle displacement or particle amplitude (ξ) is a measurement of distance of the movement of a particle in a medium as it transmits a wave. In most cases this is a longitudinal wave of pressure. In the case of a sound wave traveling through air, the particle displacement is evident in the oscillations of air molecules with, and against, the direction in which the sound wave is traveling. A particle of the medium undergoes displacement according to the particle velocity of the wave traveling through the medium, while the sound wave itself moves at the speed of sound, equal to 343 m/s in air at 20° C.

The instantaneous particle displacement ξ in m for a wave is:

ξ=∫ₜvdt

wherein:

v is velocity; and

t is time.

This expression for ξ undergoes simple harmonic oscillation, and as such is usually expressed as an RMS (root mean square) time average.

Particle displacement for a traveling wave containing a single frequency can be represented in terms of other measurements:

$$ξ = \frac{v}{ω} = \frac{p}{{Z}_{0} \cdot ω} = \frac{a}{{ω}^{2}} = \frac{1}{ω} \sqrt{\frac{1}{{Z}_{0}}} = \frac{1}{ω} \sqrt{\frac{E}{ρ}} = \frac{1}{ω} \sqrt{\frac{{P}_{ac}}{{Z}_{0} \cdot A}}$$

wherein:
a—particle acceleration (m/s²)
I—sound intensity (W/m²)
E—sound energy density (W·s/m³)
P_{ac}—acoustic power (W).

In the above equation, the quantities ξ, v, a, I, E, and P_{ac} may be taken throughout as RMS time-averages (or all as maximum values). The single frequency traveling wave has acoustic impedance equal to the characteristic impedance, Z=Z₀. Further representations for ξ can be found from the above equations using the replacement ω=2πf.

The NG Shield device is herein shown working in 30-100 Hz frequency range. Acoustic pressure measurements at 30-100 Hz frequency using Hydrophone needles method is complicated and not sensitive, as hydrophone needles are not sensitive. Therefore we chose a well known measurement method for when vibration amplitudes and frequency are measured using non contact methods and further to calculate the pressure amplitude value.

Vibration amplitudes and frequency measurement on a NG tube with a NG Shield attached was conducted with MTI-2000 Fotonic Sensor.

The MTI-2000 offers features and performance improvements that meet the applications in the measurement of displacement and vibration. It sets new performance standards with resolution to 0.01 micro inch (2.5 angstroms) and frequency response from direct coupled (dc) to 150 kHz. The measurements' set up is shown in FIG. 26. The measurements were performed at points A, B and C as shown in FIG. 25. The NG tube is represented by 25-1 and the NG Shield actuator is represented by 25-2; displacement amplitudes and calculated pressure values were measured at measurement points A, B and C.

Pressure values created due to acoustic lubrication on the NG tube were calculated as per equations above.

### Experimental Results

Results are shown in FIG. 25: Maximum displacement at (A)=0.002 mm; Maximum displacement at (B)=0.001 mm; Maximum displacement at (C)=0.0005 mm; Pₘₐₓ at (A)=1.5 kPa; Pₘₐₓ at (B)=0.9 kPa; and Pₘₐₓ at (C)=0.5 kPa.

The amplitudes of acoustic lubrication vibrations created on the nasal-gastric catheter with NG Shield device were in the range between: 0.002-0.0005 mm, when distancing 10-70 cm from the actuator. Maximum pressure created with NG Shield on NG tube (when complex of vibration frequencies consist of about 100 Hz and 30 Hz) was 1.5 kPa. The device was proved to be safe in in vitro animal tests and clinical trials.

### Example 3

### Evaluation of Relative “Friction” Between a Preload Surface and a NG Tube

The NG shield device was tested in an independent laboratory (Harland Medical Systems Ltd., using their FTS 5000 friction test system). The test system is specifically designed to measure lubricious coating performance on catheters, guide wires, pacing leads and other similar medical devices. A series of tests were undertaken to evaluate the relative “friction” between a preload surface and a tube with and without the application of acoustic lubrication and with and without the use of a lubricant (water). A preload surface is the surface area wherein a load of 150 g is in touch with NG tube. These measurements are representative of the “friction” between the surface of a medical tube and a vital tissue.

### Experimental Procedure

Data were generated by testing NG tube sections cut from a PVC (Polyvinyl Chloride) 18 Fr size NG tube manufactured by Pennine Company, US. Samples were cut from the longer intact device to accommodate the depth of the water chamber of the Harland measurement system. All samples were 30 cm long. The Harland measurement system is designed to grip a test specimen with a controlled load (in this case 150 grams) and to then pull the test sample through the grips at a controlled rate while simultaneously recording the pull force. The coefficient of friction (μ) is the Force to move the device through the grips (Fpull) divided by the Grip Force (Fgrip).

### Experimental Results

FIG. 27 presents data of the impact of acoustic lubrication applied to a NG tube. Review of the data set indicates that the maximum pull force occurred using dry conditions without the application of acoustic lubrication (210.2 grams was the pull force needed to move the sample), wherein the coefficient of friction was μ=1.4. The minimum pull force occurred using wet conditions with the application of acoustic lubrication (165:5 was the pull force needed to move the sample, wherein the coefficient of friction was μ=1.1).

Most importantly, application of acoustic lubrication to the NG tubes reduced the coefficient of friction in this model by 26%, as shown in FIG. 27.

### Example 4

### Clinical Evaluation of the Safety and Effectiveness of the NanoVibronix NG Shield Device

A clinical study on healthy volunteers was performed to evaluate the safety and effectiveness of the NanoVibronix NG Shield device in patients who require NasoGastric tube insertion.

### Experimental Procedure

The study was designed to demonstrate the effectiveness of the NG Shield device on pain and/or discomfort at the nose area (nose and face) and at the pharynx. These areas have been reported in the literature as the areas that are most painful when using a NasoGastric tube. In addition there have also been reports on tissue injuries within these areas related to NG tube usage.

The purpose of using a NG Shield device was to reduce the friction between the NG tube and the tissues surrounding it, resulting in less pain and fewer injuries for the patient. Furthermore, the low frequency ultrasound was expected to enhance healing of injuries, if injuries were to occur, due to the therapeutic effect of low intensity ultrasound.

This study used a multiple crossover design to test the hypothesis that changing the friction using acoustic lubrication produced by the NG tube will lead to changes in the pain and/or discomfort levels.

The study tested the effect of the device on young, healthy volunteers. This afforded a greater degree of control of the subjects and a greater degree of comparison of the groups. In addition, this population served as a “false positive” group model since hospitalized or chronic patients with NG tubes are typically much older and suffer from additional symptoms associated with NG tube insertion. Such patients might be more sensitive to pain or discomfort, especially when longer intubation is required.

When using healthy volunteers in a study to test pain and discomfort level, it is important to remember that a certain proportion of the group will report a low or almost no pain or discomfort at all, which may affect the results since the level of alteration that represents the effectiveness of the device is very low.

### Experimental Results

Results were tested in sub-group analyses. Pain and/or discomfort were measured using a scale of 0-10, wherein 0 represented no pain and 10 represented extreme pain. The results are presented graphically in FIG. 28, discomfort level during indwelling phase when the discomfort level in the nose or throat was at least 4, and FIG. 29, pain level during indwelling phase. The results presented in FIG. 28 show a reduction of 40-70% in the discomfort level at the nose and throat areas.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An apparatus for generating vibrations along a surface of an indwelling medical device that is in contact with a vital tissue, said apparatus comprising an actuator electrically connected to an electronic driver, wherein said driver powers said actuator;
a. wherein said actuator comprises at least one vibration element;
b. wherein said at least one vibration element produces vibrations comprising Hz range cylindrical surface vibrations at about 30-200 Hz, kHz range surface acoustic wave (SAW) vibrations at about 90-120 kHz, or a combination thereof;
c. wherein said vibrations create an acoustic lubrication effect along said surface of said indwelling medical device that is in contact with said vital tissue; and
d. wherein said acoustic lubrication effect reduces friction at an interface between the indwelling medical device and the vital tissue of a subject.

2. The apparatus according to claim 1, wherein said at least one vibration element comprises a piezo mechanical vibrator, an electro mechanical vibrator or a combination thereof.

3. The apparatus according to claim 2, wherein said piezo mechanical vibrator creates vibrations at about 90-120 kHz or at about 30-70 Hz or a combination thereof, and said electro mechanical vibrator creates vibrations at about 70-200 Hz.

4. The apparatus according to claim 1, wherein said vibrations comprise vibrations of about 30 Hz, of about 100 Hz and of about 100 kHz.

5. The apparatus according to claim 1, wherein said Hz range vibrations create an acoustic lubrication effect with an amplitude between about 1 microns to 10 microns and said kHz vibrations create an acoustic lubrication effect with an amplitude between about 0.1 micron and 1 micron.

6. The apparatus according to claim 1, wherein said actuator is disposable.

7. The apparatus according to claim 1, wherein said actuator is attached to said indwelling medical device

8. The apparatus according to claim 7, wherein said attached actuator may be repositioned while in use by sliding the actuator along the indwelling medical device either proximally or distally to an invasive end of said indwelling medical device.

9. The apparatus according to claim 7, wherein said attachment is via a clip-on stabilization mechanism.

10. The apparatus according to claim 1, wherein said indwelling medical device comprises a nasal-gastric (NG) tube, a colonoscope, a gastroscope, a duodenscope, a bronchoscope, a cytoscope, a cystoscope, a urethroscope, a hemorrhoids treatment tube, a vaginal tube, an ultrasound scope, a catheter, a cauterizing tube, a cannula, a flexible endoscope or any other medical device utilized during minimally invasive procedures.

11. A system comprising:
a. the apparatus of claim 1, said apparatus comprising an actuator electrically connected to an electronic driver, wherein said driver powers said actuator, wherein said actuator comprises at least one vibration element; wherein said at least one vibration element produces vibrations comprising Hz range cylindrical surface vibrations at about 30-200 Hz, kHz range surface acoustic wave (SAW) vibrations at about 90-120 kHz, or a combination thereof; wherein said vibrations create an acoustic lubrication effect along said surface of said indwelling medical device that is in contact with said vital tissue; and said acoustic lubrication effect reduces friction at an interface between the indwelling medical device and the vital tissue of a subject; and
b. an indwelling tube, a display, a computer, a user board, an imaging system, handles, fixtures, stickers, adhesion pads, stands, timers, detectors, sensors, attachments or a combination thereof.

12. An apparatus for generating micro-vibrations within a medical shoe pad, said apparatus comprising an actuator and a driver,
a. wherein said driver is electrically connected to said actuator;
b. wherein said driver powers said actuator;
c. wherein said actuator produces micro-vibrations;
d. wherein said micro-vibrations comprise Hz range vibrations, kHz range surface acoustic wave (SAW) vibrations, or a combination thereof; and
wherein said micro-vibrations enhance tactile sensation in a foot.

13. The apparatus according to claim 12, wherein said actuator comprises a piezo mechanical vibrator.

14. The apparatus according to claim 12, wherein said apparatus is incorporated into a medical shoe pad.

15. A medical shoe pad comprising an apparatus of claim 12 for generating micro-vibrations within said medical shoe pad, said apparatus comprising an actuator and a driver,
a. wherein said driver is electrically connected to said actuator;
b. wherein said driver powers said actuator;
c. wherein said actuator produces micro-vibrations;
d. wherein said micro-vibrations comprise Hz range vibrations, kHz range surface acoustic wave (SAW) vibrations, or a combination thereof; and
wherein said micro-vibrations enhance tactile sensation in a foot in contact with the medical shoe pad.

16. The apparatus according to claim 13, wherein said piezo mechanical vibrator creates vibrations at about 90-120 kHz or at about 30-70 Hz or a combination thereof.

17. A method for generating an acoustic lubrication effect along a surface of an indwelling medical device that is in contact with a vital tissue, said method comprising use of an apparatus of claim 1 attached to indwelling tube,
a. wherein said apparatus comprises an actuator electrically connected to an electronic driver, wherein said driver powers said actuator; wherein said actuator comprises at least one vibration element; wherein said at least one vibration element produces vibrations comprising Hz range cylindrical surface vibrations at about 30-200 Hz, kHz range surface acoustic wave (SAW) vibrations at about 90-120 kHz, or a combination thereof;
b. wherein said use of the apparatus is for generating Hz range cylindrical surface vibrations, kHz range surface acoustic wave vibrations or a combination thereof along said surface of said indwelling medical device; and
c. wherein said vibrations create an acoustic lubrication effect along said surface of said indwelling medical device that is in contact with said vital tissue; and said acoustic lubrication effect reduces friction at an interface between the indwelling medical device and the vital tissue of a subject.

18. The method of claim 17, wherein said at least one vibration element comprise a piezo mechanical vibrator, an electro mechanical vibrator or a combination thereof; wherein said piezo mechanical vibrator creates vibrations at about 90-120 kHz and/or 30-70 Hz and said electro mechanical vibrator creates vibrations at about 70-200 Hz and wherein the amplitude of said Hz range vibrations ranges between about 1 microns to 10 microns; and wherein the amplitude of said kHz range surface acoustic waves vibrations ranges between about 0.1 micron and 1 micron.

19. The method according to claim 17, wherein said attached actuator may be repositioned while in use by sliding the actuator along the indwelling medical device either proximally or distally to an invasive end of said indwelling medical device.

20. The method of claim 17, wherein said attachment is via a clip-on stabilization mechanism.

21. The method of claim 17, wherein said indwelling medical device comprises a nasal-gastric (NG) tube, a colonoscope, a gastroscope, a duodenscope, a bronchoscope, a cytoscope, a cystoscope, a urethroscope, a hemorrhoids treatment tube, a vaginal tube, an ultrasound scope, a catheter, a cauterizing tube, a cannula, a flexible endoscope or any other medical device utilized during minimally invasive procedures.

22. The method of claim 17, wherein said acoustic lubrication effect reduces pain and/or discomfort in said subject, and wherein said pain and/or discomfort is reduced during insertion, removal, indwelling or any combination thereof, of said indwelling medical device.

23. The method of claim 17, wherein said acoustic lubrication effect
a. provides a means for reducing procedural time for minimally invasive medical procedures;
b. provides a means for increasing efficiency and/or safety of use of said indwelling medical device;
c. decreases impedance to electro cautery in said vital tissue;
d. improves optical definition of structures imaged using said indwelling device; or
e. improves a quality of action of an implement that inserts into an inner channel of said medical indwelling device, wherein said implement comprises a polypectomy snare, a sphincterotome, a papillotome, a needle knife papillotome, or any other implement used for a trans-endoscopic electro surgery and wherein improvement leads to minimal injury of tissue; or any combination thereof.

24. The method of claim 23, wherein said minimally invasive medical technique comprises a naso-gastric tube insertion; an endoscopic procedure comprising a colonoscopy, imaging technique, hemorrhoid treatment, trans-endoscopic electro surgery; insertion of a said medical indwelling device; frigidity treatment; or any combination thereof.
